(19) United States
(12) Patent Application Publication
(10) Pub. No.: US 2008/0241150 A1
(43) Pub. Date: Oct. 2, 2008

(54) Functional negative regulatory domain sequences from human NOTCH1 and 2 and isolated LNR domains from human NOTCH1

(75) Inventors: Stephen Blacklow, Cambridge, MA (US); Didem Vardar-Ulu, Roslindale, MA (US); Wendy Ryan Gordon, Jamaica Plain, MA (US); Cheryll Sanchez-Irizarry, San Mateo, CA (US); Jon C. Aster, Lexington, MA (US)

Correspondence Address: WOLF GREENFIELD & SACKS, P.C., 600 ATLANTIC AVENUE, BOSTON, MA, 02210-2206, US

(73) Assignee: The Brigham and Women's Hospital, Inc., Boston, MA (US)

(21) Appl. No.: 11/803,705

(22) Filed: May 15, 2007

Related U.S. Application Data
(60) Provisional application No. 60/800,340, filed on May 15, 2006.

(51) Int. Cl. A61K 39/395; C07K 14/00; A61K 31/70; C12P 21/04; C07H 21/04; G01N 33/53; C12N 5/00; C07K 16/00

(52) U.S. Cl. 424/139.1; 530/300; 514/44; 530/387.9; 435/375; 435/69.6; 435/7.1; 536/23.5

(57) ABSTRACT

The present invention relates to a LNR-HD domain of the Notch receptor and methods of use. The method for expression and structural determination of the LNR-HD domain is described.

### RELATED APPLICATIONS

This application claims priority under 35 U.S.C. § 119(e) to U.S. Provisional Application Ser. No. 60/800,340, filed on May 15, 2006, which is herein incorporated by reference in its entirety.

### FEDERALLY SPONSORED RESEARCH

This invention was made with Government support under NIH grant number R01 CA92433. Accordingly, the Government may have certain rights in this invention.

### FIELD OF THE INVENTION

The present invention relates to a LNR-HD domain of the Notch receptor and methods of use thereof. Methods for expression and structural determination of the LNR-HD domain are also described.

### BACKGROUND OF THE INVENTION

Notch proteins define a unique class of highly conserved transmembrane receptors regulating cell growth, differentiation, and death in different tissues of multicellular organisms (Artavanis-Tsakonas et al., 1999, Science 284, 770-776). The first human homologue of NOTCH1 was identified through its involvement in chromosomal translocations found in human T-cell Acute Lymphoblastic Leukemias/Lymphomas (T-ALL) (Ellisen et al., 1991, Cell 66, 649-661). Notch receptors are first synthesized as 300-350 kDa type I single-pass transmembrane glycoproteins. During maturation, Notch precursor polypeptides are proteolytically processed by a furin-like convertase at a site called S1 (FIG. 1). The resulting two associated subunits, here termed extracellular Notch (N^{EC}) and transmembrane Notch (N^{TM}), constitute the mature heterodimeric form of the protein present at the cell surface (Sanchez-Irizarry et al., 2004, Mol. Cell. Biol. 24, 9265-9273). The N^{EC} subunit contains EGF like repeats that include the region responsible for ligand binding. The EGF repeats are followed by a negative regulatory region (NRR) that participates in restraining premature activation of Notch receptors (Rand et al., 2000, Mol. Cell. Biol. 20, 1825-1835). The NRR includes three Notch-specific Lin12-Notch repeats (LNRs) and the heterodimerization domain (HD), which contains an additional 103-residue sequence up to the furin cleavage site (S1) as well as the extracellular portion of the N^{TM} subunit. The transmembrane segment of N^{TM} is followed by the intracellular region (ICN) that consists of a RAM domain, seven ankyrin repeats (ANK), a transactivation domain (TAD) and a C-terminal PEST region.

The signaling of Notch receptors is mediated by ligand binding to the extracellular domain. Proteins of the DSL family (Delta, Serrate and Lag-2) are ligands that can bind to the EGF repeats in the extracellular domain, thereby initiating Notch activation. Upon binding of the ligands the N^{TM} subunit gets cleaved by a metalloprotease at S2, a site just external to the transmembrane domain (Mumm et al., 2000, Mol. Cell. 5, 197-206). This cleaved product (N^{TM}*) is subsequently proteolyzed again by an enzyme complex called gamma-secretase, with the final intramembrane cleavage occurring just internal to the inner membrane leaflet. Gamma-secretase cleavage results in release of the ICN. Upon its release, ICN travels to the nucleus and interacts with members of the CSL family of transcription factors, mediated through binding of the RAM and ANK fragments (Nam et al., 2003, J. Biol. Chem. 278, 21232-21239). Binding of ICN to CSL results in recruitment of proteins of the Mastermind family, and this ternary complex engages proteins like p300 and other factors to activate transcription of a variety of target genes. However, in the case of Notch1, the complex is short lived and is targeted for degradation by a C-terminal destruction box in its C-terminal PEST region (Aster, 2005, Int. J. Hematol. 82, 295-301).

Enforced expression of forms of Notch lacking the N^{EC} subunit results in constitutively active Notch signaling in a variety of model organisms (Rebay et al., 1993, Cell 74, 319-329). In contrast, forms lacking the EGF-like repeats but retaining the NRR, are maintained in an inactive state and are unable to respond to the ligand. Therefore, the major role of the NRR region within the context of the full-length Notch receptor is to prevent activation of Notch receptors in the absence of ligands, yet permit activation in response to ligand binding (Sanchez-Irizarry et al., 2004, Mol. Cell. Biol. 24, 9265-9273). Within the NRR region, the LNR domains are responsible for preventing proteolysis in the absence of ligand-binding while the HD region is responsible for heterodimerization (i.e. holding the two furin-cleaved subunits together). Each LNR module consists of 35-40 residues, has at least two disulfide bonds, and is likely to have a tertiary structure resembling that of LNR-A from hNotch1 solved by solution NMR spectroscopy (Vardar et al., 2003, Biochem. 42, 7061-7067).

The hNotch1 receptor and a number of its substructures have been expressed in mammalian cells to elucidate the function of the specific structural elements (Sanchez-Irizarry et al 2004 Moll Cell Biol 23, 9265-9273). Expression of Notch receptors lacking the EGF repeats and LNR domain but retaining the HD region resulted in a constitutively active Notch receptor. Addition of the LNR region resulted in inactivation of the receptor. Therefore, the LNR region prevents the second proteolysis step at S2, thereby preventing further processing of S3 and the generation of the ICN unit that can translocate to the cell nucleus.

### SUMMARY OF THE INVENTION

The present invention relates to a LNR-HD domain of the Notch receptor and methods of use.

In one aspect, an isolated peptide comprising a human LNR-HD domain having at least one deletion or substitution from a native LNR-HD domain, wherein the isolated peptide has a native conformation, is provided. In an embodiment the LNR-HD domain is a hNotch1 LNR-HD domain or a hNotch2 LNR-HD domain. The LNR-HD domain peptide may be produced through recombinant DNA technology and may contain sequences or elements that facilitate expression of the isolated peptide from recombinant DNA. In some embodiments the peptide has the sequence of any one of SEQ ID NOs 1-16.

In another aspect, the invention is an isolated peptide consisting of a human LNR-HD domain, wherein the isolated peptide has a native conformation.

In other aspects, the invention further embraces LNR-HD domain peptides, wherein the peptide has one or more mutations associated with T-cell acute lymphoblastic leukemia/lymphoma. In some embodiments the peptide has a native conformation.

Another aspect of the invention provides a method for treating a disease comprising administering to a subject in need of such a treatment an effective amount for treating the disease of a nucleic acid of at least 15 nucleotides which hybridizes under stringent conditions to a LNR-HD domain. In an embodiment, the method pertains to LNR-HD domains containing one or more mutations associated with T-cell acute lymphoblastic leukemia/lymphoma. The nucleic acid to be administered may be an siRNA, antisense RNA or antisense DNA. A variety of diseases may be treated including cancer, in particular T-cell acute lymphoblastic leukemia/lymphoma, an immune or inflammatory disorder like colitis or asthma, an infectious disease, an angiogenesis disorder, atherosclerosis, or a disorder of the kidney including glomerulonephritis.

In another aspect, the invention describes compounds comprising an LNR-HD antibody or fragment thereof that can bind to any of the LNR-HD domain peptides. In an embodiment the LNR-HD antibody or fragment thereof does not bind to a region of the hNotch receptor other than the LNR-HD domain

In another aspect the invention is a compound including an LNR-HD antibody or fragment thereof that binds to a LNR-HD domain but does not bind to a region of the hNotch receptor other than the LNR-HD domain.

The invention in another aspect is a method for treating a disease comprising administering to a subject in need of such a treatment an effective amount for reducing Notch activity of an LNR-HD antibody or fragment thereof. In one embodiment, the LNR-HD domain may contain one or more mutations associated with T-cell acute lymphoblastic leukemia/lymphoma. In another embodiment, the LNR-HD antibody or fragment thereof binds to the native hNotch1 or hNotch2 receptor and abolishes or attenuates the function of the native hNotch1 or hNotch2 receptor. In yet another embodiment, the antibody or fragment thereof may bind to the LNR domain. In a further embodiment, the LNR-HD antibody or fragment thereof binds to an LNR-HD domain but does not bind to a region of the hNotch receptor other than the LNR-HD domain. A variety of diseases may be treated with the above described methods including cancer, in particular T-cell acute lymphoblastic leukemia/lymphoma, an immune or inflammatory disorder like colitis or asthma, an infectious disease, an angiogenesis disorder, atherosclerosis, or a disorder of the kidney including glomerulonephritis.

Another aspect of the invention is a method for promoting stem cell survival comprising contacting a stem cell with an effective amount for promoting cell survival of an LNR-HD antibody or fragment thereof. In one embodiment of the method for promoting stem cell survival, the antibody binds to the native hNotch1 or hNotch2 receptor and induces or stimulates the function of the native hNotch1 or hNotch2 receptor. In another embodiment of the method for promoting stem cell survival, the antibody binds to the HD domain.

The invention also provides in another aspect methods for producing an LNR-HD antibody including, contacting an antibody producing cell with an isolated peptide comprising a human LNR-HD domain that is not in the context of the full length hNotch protein, under conditions effective to produce a LNR-HD specific antibody producing cell, and promoting production of the LNR-HD antibody by the antibody producing cell. These methods may include LNR-HD domains that contain one or more mutations associated with T-cell acute lymphoblastic leukemia/lymphoma.

Another aspect of the invention is methods for treating a disease comprising administering to a subject in need of such a treatment an effective amount of isolated LNR-HD domain peptides. These methods may involve binding of the isolated peptides to the native hNotch1 or hNotch2 receptor, which may contain one or more mutations associated with T-cell acute lymphoblastic leukemia/lymphoma. In a further embodiment, the method for treating a disease may involve binding of the isolated peptide to the LNR-HD domain, which may contain mutations associated with T-cell acute lymphoblastic leukemia/lymphoma. In yet another embodiment, the isolated peptides used for treating the disease have been modified to increase the half-life and effectiveness of the isolated peptides.

The invention also provides in another aspect a method for identifying a putative therapeutic compound, comprising contacting an isolated LNR-HD domain peptide with a compound to determine if the compound binds to the isolated peptide, wherein if the compound binds to the isolated peptide the compound is a putative therapeutic compound. The compound may be an agonist, antagonist or inhibitor. In another embodiment, the invention embraces a method for identifying a chemical compound which specifically binds to the isolated LNR-HD domain peptide, which comprises contacting cells containing DNA encoding and expressing on their cell surface the isolated LNR-HD domain peptide, with the compound under conditions suitable for binding, and detecting specific binding of the chemical compound to the LNR-HD domain peptide. The compound may be an agonist, antagonist or inhibitor

The invention in another aspect is a method for engineering Notch constructs by substituting or deleting one or more amino acid residues in the region of the HD domain flanking the furin cleavage site based on sequence homology and structural data to produce an isolated peptide in its native structure with native disulfide bond connectivity.

In another aspect, the invention describes an isolated nucleic acid encoding a peptide comprising a human LNR-HD domain having at least one deletion or substitution from a native LNR-HD domain, wherein the nucleic acid is cDNA, genomic DNA or RNA. The LNR-HD domain may contain one or more mutations associated with T-cell acute lymphoblastic leukemia/lymphoma.

The invention also includes in another aspect a vector which contains the nucleic acid sequence, and which comprises the elements necessary for expression of the peptide in bacteria, yeast, insect cells or mammalian cells.

A further aspect of the invention pertains to kits. The invention is a kit comprising a container housing an LNR-HD domain therapeutic and instructions for administering the components in the kit to a subject at risk of, or in need of, treatment of a disease. The LNR-HD therapeutic may be an LNR-HD peptide, an LNR-HD antibody, an LNR-HD nucleotide. The LNR-HD therapeutic may also be an agonist, antagonist or inhibitor of the LNR-HD domain. The kit may further comprise a container housing a pharmaceutical preparation diluent. A variety of diseases may be treated with the LNR-HD domain therapeutic including cancer, in particular T-cell acute lymphoblastic leukemia/lymphoma, an immune or inflammatory disorder like colitis or asthma, an infectious disease, an angiogenesis disorder, atherosclerosis, or a disorder of the kidney including glomerulonephritis.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing”, “involving”, and variations thereof herein, is meant to encompass the items listed thereafter and equivalents thereof as well as additional items.

### BRIEF DESCRIPTION OF DRAWINGS

FIG. 1 shows an overview of Notch protein domain organization.

FIG. 2 shows hNotch2 LNR-HD crystals (pN2FL-del; Seq ID No 16). Crystallization conditions: 100 mM Bis Tris pH 6.5, 200 mM MgCl₂, 18% PEG 3350, 10% glycerol.

FIG. 3 shows hNotch2 LNR-HD crystals (pN2FL-del; Seq ID No 16) diffraction to 2.5 Angstroms.

FIG. 4 shows hNotch2 LNR-HD crystals (pN2FL-del; Seq ID No 16) with electron density map and initial model.

FIG. 5 depicts a kit with LNR-HD domain therapeutics (10=kit, 12=LNR-HD domain therapeutic; 14=additional components; 20=instructions).

FIG. 6 provides the Notch domain organization and overall views of the hNotch2 three-dimensional structure. (a) Overview of Notch signaling. Ligand binding to the extracellular portion of Notch triggers metalloprotease cleavage at site S2. The resulting truncated transmembrane subunit of the receptor is a substrate for cleavage at S3 by gamma-secretase, which releases the intracellular part of Notch (ICN) from the membrane. ICN migrates to the nucleus, where it assembles into a complex that turns on transcription of target genes. The structure reported is of the Negative Regulatory Region, highlighted in the box. (b) Two views of the NOTCH2 NRR related by a 90-degree rotation are shown. In the ribbon representation of the NRR, the LNR modules are colored different shades and the HD domain is colored in two shades; the light and dark represent residues N- and C-terminal to the furin cleavage loop, respectively. The positions of S1 and S2 cleavage are indicated with arrows. (c) Sequence conservation mapped onto the NRR structure, colored according to sequence conservation (see FIG. 10 for sequence alignment). The LNR modules are rendered as a molecular surface, and the HD domain is rendered in ribbon representation. Side chains in the hydrophobic core of the HD domain, defined as residues within the α/β sandwich having less than 10% solvent accessibility, are shown in ball and stick form. (d) Sites of tumor-associated NOTCH1 HD domain mutations mapped onto the NOTCH2 NRR structure. The LNRs are shown as a surface, and the HD domain backbone as a ribbon. Hydrophobic core residues from FIG. 6c are shown in ball and stick form and side-chains of residues corresponding to tumor-associated mutations are colored dark.

FIG. 7 provides a comparison of the human NOTCH2 HD domain with SEA domains from mucins. a. HD domain from human NOTCH2. Residues N- and C-terminal to the furin cleavage site are colored light cyan and teal, respectively. b. SEA domain from Mucin-1 (pdb: 2acm). Residues N- and C-terminal to the autoproteolysis site are colored light orange and maroon, respectively. c. SEA domain from Mucin-16 (pdb: livz), colored purple. The loop from Mucin-16 that aligns with the sequence of the autoproteolysis site from Mucin-1 is indicated.

FIG. 8 shows the interface between the LNR and HD domains. (a) LNR-HD contact interface. The LNR domain surface is colored dark gray when an atom approaches within 4 Å of the HD domain, and light gray elsewhere. The HD domain is colored dark when an atom approaches within 4 Å of the LNR domain, and light elsewhere. (b) Anchoring of helix 3. Helix 3 is clamped in position above the S2 site by a hydrophobic interface with residues from LNR-B and the LNR-AB linker and a conserved hydrogen bond from LNR-A. (c) The LNR-AB linker sterically blocks access to the metalloprotease cleavage site. The left panel shows the hydrophobic pocket in the HD domain that houses the S2 site in a surface representation and resides from the LNR-AB linker in ball-stick representation. The right panel is a view down the scissile bond cleaved by metalloprotease, which has been rotated about 90 degrees from the left view. The residues in the β-strand containing the S2 cleavage site are shown in ball and stick form and three ‘gatekeeper residues’ from the LNR-AB linker are in surface representation.

FIG. 9 depicts the results of a cell-based reporter gene assays showing that stepwise removal of LNR domain elements causes ligand-independent activation of NOTCH. Luciferase assays were performed in triplicate on U20S cell lysates prepared from cells transfected with 10 ng of plasmids encoding the indicated forms of (a) a NOTCH2/NOTCH1 chimera or (b) NOTCH1, along with a luciferase reporter plasmid containing iterated CSL-binding sites, and an internal control plasmid expressing Renilla luciferase. Firefly luciferase activity from cell lysates was measured in triplicate, normalized, and expressed relative to the activity in extracts prepared from cells transfected with the AEGF NOTCH2/NOTCH1 chimera (a) or ΔEOF NOTCH1 (b). +/− error bars represent the standard deviations of the measurements.

FIG. 10 shows the sequence alignment of the NRR region of various Notch receptors, colored according to sequence conservation; dark-absolutely conserved, lighter-highly conserved (defined by Clustal-W strong conservation groups and/or >80% sequence identity), lightest-moderately conserved (defined by Clustal-W weak conservation groups or >50% sequence identity), white-non-conserved. Amino acid residues of special importance are denoted as follows: side-chain and main-chain Ca⁺⁺-coordinating residues, circles and triangles, respectively; Zn⁺⁺-coordinating residues, squares; residues mutated in NOTCH1 in T cell acute lymphoblastic leukemia, asterisks.

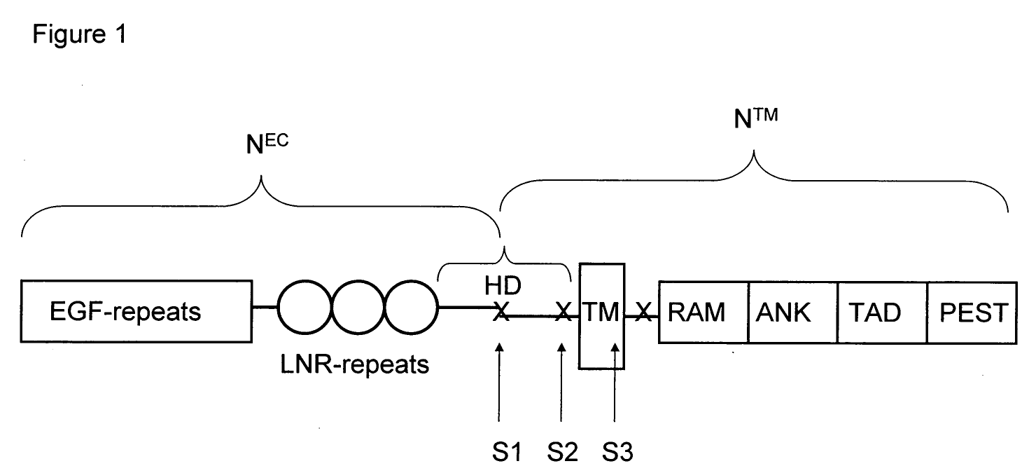

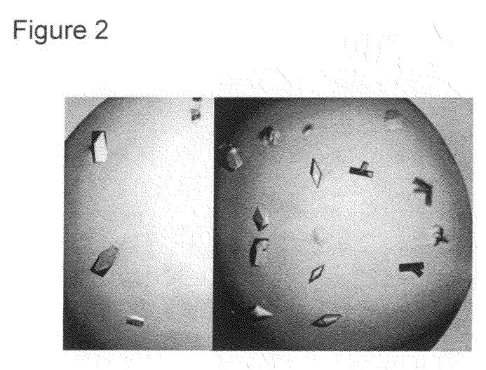

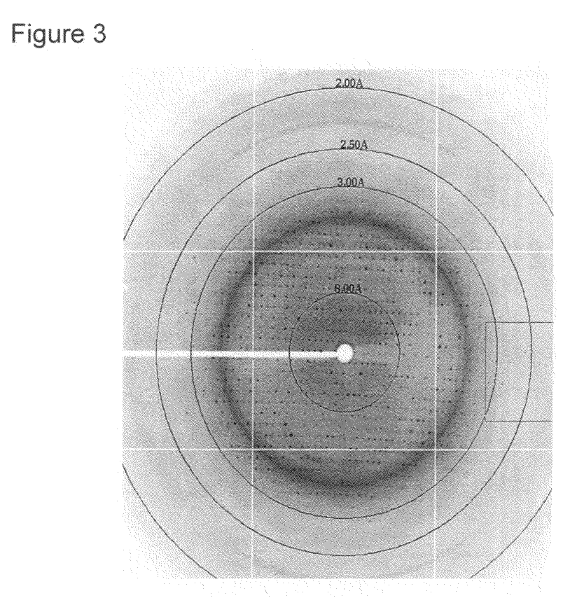

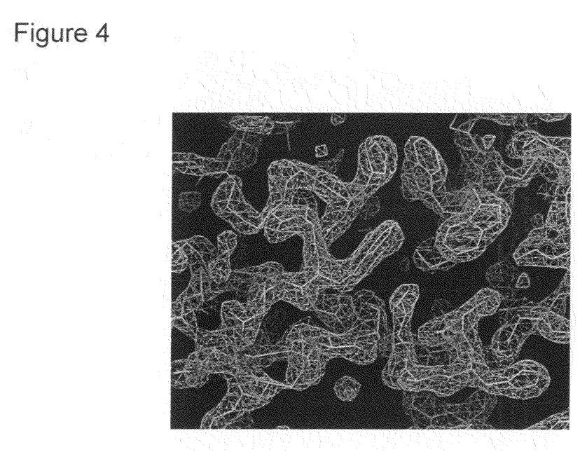

### DETAILED DESCRIPTION

Notch receptors participate in a conserved signaling pathway that regulates many different cellular differentiation events, including regulation of cell fate and the capacity to promote proliferation or death (Bray et al., 1998, Cell. Dev. Biol. 9, 591-597). These diverse functions are mediated through a signal transduction pathway involving regulated proteolysis of Notch receptors. During transport to the cell surface the receptors are modified and cleaved by a furin-like protease at site S1˜69 residues N-terminal to the membrane resulting in mature heterodimeric receptors consisting of non-covalently associated extracellular (N^{EC}) and transmembrane units (N^{TM}). The N^{EC} domain contains a series of about 30 EGF like repeats that bind DSL (Delta-Serrate-Lag1) ligands, three LNR repeats (Lin-12 Notch repeats) and a conserved heterodimerization domain (HD) (Sanchez-Irizarry et al., 2004; Pear et al., 2004, Curr. Opin. Hematol. 11, 426-433) which maintains stable association of the N^{EC} and N^{TM} subunits. The intracellular portion of N^{TM} (ICN) contains a RAM region, seven ankyrin repeats, and a C-terminal PEST sequence. The structural building blocks of all 4 human Notch receptors are similar, with the main differences being in the number of EGF repeats or in the sequence of the intracellular region between the ankyrin repeats and the PEST sequence.

The LNR-HD domain comprises the three Notch-specific Lin12-Notch repeats (LNRs) and the heterdimerization region (HD) up to the membrane. The LNR-HD domain is an important component of normal Notch signaling and its isolation offers the potential of intervening in the Notch receptor pathway. If the LNR-HD domain could be ‘locked’ in a conformation that would no longer allow the cleavage of the ICN domain, the function of the Notch receptor would be abrogated. On the other hand, if the LNR-HD domain could be converted from its native conformation into a form permissive for S2 cleavage, activation of the Notch receptor would result.

To unlock the therapeutic potential of the LNR-HD domain, the structure of the LNR-HD domain has been elucidated. The invention presented herein discloses the materials and methods that were used to determine the structure and mechanism of function of the LNR-HD domain. The invention also encompasses compositions and methods of use of native LNR-HD domains as well as modified LNR-HD domains having at least one deletion or substitution from a native LNR-HD domain. As used herein, a native LNR-HD domain is a naturally occurring form of LNR-HD including three connected LNR domains and the heterodimerization domain. The native LNR-HD domain may have the sequence of any naturally occurring notch receptor, but preferably has the sequence of a human notch receptor. Human notch receptors include, for instance, hNotch1, hNotch2, hNotch3 and hNotch4.

The LNR-HD domain may be an isolated peptide. An isolated peptide or molecule is a molecule that is substantially pure and is free of other substances with which it is ordinarily found in nature or in vivo systems to an extent practical and appropriate for its intended use. In particular, the molecular species are sufficiently pure and are sufficiently free from other biological constituents of host cells so as to be useful in, for example, producing pharmaceutical preparations or sequencing if the molecular species is a nucleic acid, peptide, or polysaccharide. Because an isolated molecular species of the invention may be admixed with a pharmaceutically-acceptable carrier in a pharmaceutical preparation or be mixed with some of the components with which it is associated in nature, the molecular species may comprise only a small percentage by weight of the preparation. The molecular species is nonetheless substantially pure in that it has been substantially separated from the substances with which it may be associated in living systems.

The LNR-HD domain may be in the context of, or separate from, the full length notch protein or portions thereof. If the LNR-HD domain is in the context of the notch protein it may form a full length or partial notch receptor. The full length or partial notch receptor may include the native sequence or may include insertions, deletions or substitutions. When the peptide consists of the LNR-HD domain it is not found in the context of a partial or full length notch receptor.

Modified LNR-HD domains having at least one substitution, deletion or insertion are also useful according to the invention. As used herein, a “conservative amino acid substitution” or “conservative substitution” refers to an amino acid substitution in which the substituted amino acid residue is of similar charge as the replaced residue and is of similar or smaller size than the replaced residue. Conservative substitutions of amino acids include substitutions made amongst amino acids within the following groups: (a) the small non-polar amino acids, A, M, I, L, and V; (b) the small polar amino acids, G, S, T and C; (c) the amido amino acids, Q and N; (d) the aromatic amino acids, F, Y and W; (e) the basic amino acids, K, R and H; and (f) the acidic amino acids, E and D. Substitutions which are charge neutral and which replace a residue with a smaller residue may also be considered “conservative substitutions” even if the residues are in different groups (e.g., replacement of phenylalanine with the smaller isoleucine). The term “conservative amino acid substitution” also refers to the use of amino acid analogs or variants.

Methods for making amino acid substitutions, additions or deletions are well known in the art. The terms “conservative substitution”, “non-conservative substitutions”, “non-polar amino acids”, “polar amino acids”, and “acidic amino acids” are all used consistently with the prior art terminology. Each of these terms is well-known in the art and has been extensively described in numerous publications, including standard biochemistry text books, such as “Biochemistry” by Geoffrey Zubay, Addison-Wesley Publishing Co., 1986 edition, which describes conservative and non-conservative substitutions and properties of amino acids which lead to their definition as polar, non-polar or acidic.

The modified LNR-HD domains having at least one substitution, deletion or insertion have, in some embodiments, a native conformation. A native conformation as used herein refers to a tertiary structure that is similar to the tertiary structure of native LNR-HD domain. The tertiary structure of modified or native LNR-HD domains can be assessed using structural analysis such as crystallography or by functional analysis, such as binding and/or activity assays and NMR spectroscopy.

Crystallographic data can be obtained by performing crystallographic analysis on crystals of the LNR-HD domain. Crystals can be grown by various methods, such as, for example, sitting or hanging drop vapor diffusion. In general, crystallization can be performed at a temperature of from about 4° C. to about 60° C. The LNR-HD domain can be crystallized from a solution including NaCl, MgCl₂, Tris buffer and polyethylene glycol (PEG). The solution can include a precipitant, such as ammonium sulfate. Structural data describing a crystal can be obtained, for example, by X-ray diffraction. X-ray diffraction data for the crystals can be collected by a variety of means in order to obtain structural coordinates. Suitable X-ray sources include rotating anode and synchrotron sources (e.g., NSLS, Brookhaven, N.Y.). The X-ray diffraction data can be used to construct an electron density map of the LNR-HD domain. Creation of an electron density map typically involve using information regarding the phase of the X-ray scatter. Methods for calculating phase from X-ray diffraction data, include, without limitation, multiwavelength anomalous dispersion (MAD), multiple isomorphous replacement (MIR), multiple isomorphous replacement with anomalous scattering (MIRAS), reciprocal space solvent flattening, molecular replacement, and single isomorphous replacement with anomalous scattering (SIRAS), or a combination thereof. These methods generate phase information by making isomorphous structural modifications to the native protein, such as by including a heavy atom or changing the scattering strength of a heavy atom already present, and then measuring the diffraction amplitudes for the native protein and each of the modified cases. If the position of the additional heavy atom or the change in its scattering strength is known, then the phase of each diffracted X-ray can be determined by solving a set of simultaneous phase equations. The location of heavy atom sites can be identified using a computer program, such as SHELXS, (Sheldrick, Institut Anorg. Chemie, Gottingen, Germany) or Sharp (Global Phasing, Cambridge, UK) and diffraction data can be processed using computer programs such as MOSFLM, SCALA, SOLOMON, (“The CCP4 Suite: Programs for Protein Crystallography,” 1997, Acta Crystallogr. Sect. D, 54, 905-921; deLa Fortelle et al. 1997, Meth. Enzym. 276, 472-494) and HKL2000 (HKL Research, Charlottesville, Va.). Upon determination of the phase, an electron density map of the complex can be constructed.

The electron density map can subsequently be used to derive a representation of a polypeptide, a complex, or a fragment of a polypeptide or complex by fitting a three-dimensional model of a polypeptide or complex into the electron density map.

The conformation of the LNR-HD domain may also be assessed by whether the LNR-HD domain is able to bind to compounds that the native LNR-HD domain binds to. The binding of the LNR-HD domain to notch related molecules may be determined according to standard procedures.

In certain embodiments, antibodies or antigen-binding fragments thereof to the LNR-HD domain are also encompassed by the invention. The antibodies or fragments thereof may be selected for the ability to bind the LNR-HD domain of the Notch receptor. In further embodiments, the antibody or antigen-binding fragment thereof is selected from the group consisting of IgG1, IgG2, IgG3, IgG4, IgM, IgA1, IgA2, IgAsec, IgD, IgE or has immunoglobulin constant and/or variable domain of IgG1, IgG2, IgG3, IgG4, IgM, IgA 1, IgA2, IgAsec, IgD or IgE. In other embodiments, the antibody is a bispecific or multispecific antibody. In still other embodiments, the antibody is a recombinant antibody, a polyclonal antibody, a monoclonal antibody, a humanized antibody or a chimeric antibody, or a mixture of these. In particularly preferred embodiments, the antibody is a human antibody, e.g., a monoclonal antibody, polyclonal antibody or a mixture of monoclonal and polyclonal antibodies. In still other embodiments, the antibody is a bispecific or multispecific antibody. Preferred antigen-binding fragments include a Fab fragment, a F(ab′)₂ fragment, and a Fv fragment CDR3.

Antibodies can be raised against the full length Notch receptor or against specific domains of the Notch receptor. Antibodies can be generated by injecting an animal, preferably a rabbit or goat or mouse, with the antigen, in this case the Notch receptor or domains of the Notch receptor.

In order to prepare polyclonal antibodies, fusion proteins containing the complete or defined fragments of the Notch-LNR-HD domain protein can be synthesized in bacteria by expression of corresponding DNA sequences in a suitable cloning vehicle. The protein can then be purified, coupled to a carrier protein and mixed with Freund's adjuvant (to help stimulate the antigenic response by the rabbits) and injected into rabbits or other laboratory animals. Alternatively, protein can be isolated from cultured cells expressing the protein. Following booster injections at bi-weekly intervals, the rabbits or other laboratory animals are then bled and the sera isolated. The sera can be used directly or purified prior to use, by various methods including affinity chromatography, Protein A-Sepharose, Antigen Sepharose, Anti-mouse-Ig-Sepharose. The sera can then be used to probe protein extracts run on a polyacrylamide gel to identify the Notch-LNR-HD domain. Alternatively, synthetic peptides can be made to the antigenic portions of the protein and used to inoculate the animals.

To produce monoclonal Notch-LNR-HD antibodies, mice are injected multiple times (see above), the mice spleens are removed and resuspended in a phosphate buffered saline (PBS). The spleen cells serve as a source of lymphocytes, some of which are producing antibody of the appropriate specificity. These are then fused with a permanently growing myeloma partner cell, and the products of the fusion are plated into a number of tissue culture wells in the presence of a selective agent such as HAT. The wells are then screened to identify those containing cells making useful antibody by ELISA. These are then freshly plated. After a period of growth, these wells are again screened to identify antibody-producing cells. Several cloning procedures are carried out until over 90% of the wells contain single clones which are positive for antibody production. From this procedure a stable line of clones is established which produce the antibody. The monoclonal antibody can then be purified by affinity chromatography using Protein A Sepharose, ion-exchange chromatography, as well as variations and combinations of these techniques (See e.g. U.S. Pat. No. 6,998,467).

For antibodies to be used in therapy in humans, they preferably are ‘humanized’. Humanization of antibodies involves replacing native mouse sequences with human sequences as to lower the chance of an immune response once the therapeutic antibody is introduced into humans.

Notch receptor activation relies on ligand-induced proteolysis. Binding of DSL ligands leads to a cleavage at S2 in the extracellular portion of the N^{TM}. Further cleavage by a gamma-secretase complex at site 3 results in release of the intracellular domain of N^{TM} (ICN) allowing for translocation of this domain to the nucleus. In the nucleus, ICN forms a multiprotein complex that activates transcription of target genes by binding to transcription factors and scaffolding proteins like Mastermind-like-1-3, that recruit coactivators. Nuclear ICN is short-lived, being targeted for destruction through a mechanism involving C-terminal destruction boxes in the PEST region common to all Notch receptors.

The molecules of the invention including peptides, nucleic acids, antibodies and fragments thereof are useful in in vitro, in vivo and ex vivo methods. The LNR-HD notch domain offers a window for therapeutic intervention, because it offers the opportunity to abrogate or stimulate Notch signaling. An essential component of Notch signaling is the cleavage at S2 by metalloproteases. The access to the S2 site by the metalloprotease is restricted in the native conformation of LNR-HD. Upon ligand binding to the EGF repeats the LNR-HD changes conformation allowing access to the S2 site. The mutations found in the LNR-HD region in cancers, such as many T-ALL cell lines, fail to provide protection to the S2 site even in the absence of ligand binding. The LNR-HD could potentially be locked in its restrained conformation, thereby blocking/restricting access to site S2 and inhibiting or decreasing Notch signaling. This ‘locking’ could be achieved by a compound, including polypeptides, and antibodies. In addition to ‘locking’ these compounds and antibodies could also abrogate Notch signaling by direct blocking of access of the S2 site for metalloproteases. This therapeutic intervention can be executed in the native LNR-HD as well as in disease causing mutants.

Thus the compounds of the invention are useful for treating subjects having cancer as well as other disorders described herein. A “subject” shall mean a human or vertebrate mammal including but not limited to a dog, cat, horse, cow, pig, sheep, goat, or primate, e.g., monkey.

“Cancer” as used herein refers to an uncontrolled growth of cells which interferes with the normal functioning of the bodily organs and systems. Cancers which migrate from their original location and seed vital organs can eventually lead to the death of the subject through the functional deterioration of the affected organs. Hemopoietic cancers, such as leukemia, are able to outcompete the normal hemopoietic compartments in a subject, thereby leading to hemopoietic failure (in the form of anemia, thrombocytopenia and neutropenia) ultimately causing death.

A metastasis is a region of cancer cells, distinct from the primary tumor location resulting from the dissemination of cancer cells from the primary tumor to other parts of the body. At the time of diagnosis of the primary tumor mass, the subject may be monitored for the presence of metastases. Metastases are most often detected through the sole or combined use of magnetic resonance imaging (MRI) scans, computed tomography (CT) scans, blood and platelet counts, liver function studies, chest X-rays and bone scans in addition to the monitoring of specific symptoms.

Cancer, as used herein, includes the following types of cancer, breast cancer, biliary tract cancer; bladder cancer; brain cancer including glioblastomas and medulloblastomas; cervical cancer; choriocarcinoma; colon cancer; endometrial cancer; esophageal cancer; gastric cancer; hematological neoplasms including acute lymphocytic and myelogenous leukemia; T-cell acute lymphoblastic leukemia/lymphoma; hairy cell leukemia; chromic myelogenous leukemia, multiple myeloma; AIDS-associated leukemias and adult T-cell leukemia lymphoma; intraepithelial neoplasms including Bowen's disease and Paget's disease; liver cancer; lung cancer; lymphomas including Hodgkin's disease and lymphocytic lymphomas; neuroblastomas; oral cancer including squamous cell carcinoma; ovarian cancer including those arising from epithelial cells, stromal cells, germ cells and mesenchymal cells; pancreatic cancer; prostate cancer; rectal cancer; sarcomas including leiomyosarcoma, rhabdomyosarcoma, liposarcoma, fibrosarcoma, and osteosarcoma; skin cancer including melanoma, Kaposi's sarcoma, basocellular cancer, and squamous cell cancer; testicular cancer including germinal tumors such as seminoma, non-seminoma (teratomas, choriocarcinomas), stromal tumors, and germ cell tumors; thyroid cancer including thyroid adenocarcinoma and medullar carcinoma; and renal cancer including adenocarcinoma and Wilms tumor. Other cancers will be known to one of ordinary skill in the art.

In some embodiments the invention is particularly useful in the treatment of T-cell acute lymphoblastic leukemia/lymphoma (T-ALL). Mutations in the Notch1 receptor have been found in more than 50% of patients with T-ALL (Weng et al., 2004, Science 306, 269-271). In one rare mutation in T-ALL, a chromosomal translocation t(7;9) results in a fusion of the Notch receptor to a TCR-beta promoter/enhancer resulting in the expression of a series of truncated mRNAs driving the expression of N-terminally truncated constitutively active Notch1 polypeptides (Ellisen et al., 1991, Cell 66, 649-661). Gamma-secretase inhibitors do not inhibit the growth of these cells implying that some of the Notch1 specific peptides can access the nucleus in a gamma-secretase-independent fashion. The majority of T-ALL cells are negative for this specific translocation and have a different mechanism of activation. These cell lines do undergo cell arrest when exposed to either a gamma-secretase inhibitor or an inhibitor to the intracellular nuclear complex. Initial studies showed that most mutations are found on the HD domain, causing various non-conserved substitutions (Weng et al., 2004, Science 306, 269-271). The second set of mutations fell in the C-terminal destruction box, mostly resulting in reading frame shifts and loss of these sequences. When 96-well characterized human pediatric T-ALL samples were analyzed, most of those samples also showed mutations in the heterodimerization domain both within the C-terminal portion of NEC and the extracellular region of N^{TM} (Weng et al., 2004, Science 306, 269-271). Remarkably, a number of these mutations occur in cis with deletions of the C-terminal PEST sequences as well.

Studies on deletions of C-terminal sequences caused by retroviral insertion suggested that loss of these sequences increases notch signaling by enhancing stability of the ICN (Feldman et al., 2000, Blood 96, 1906-1013). The mutations in the heterodimerization domain lead to increased activation of the Notch receptor, presumably through increased susceptibility to cleavage by metalloproteases. The activating mutations cause the Notch1 heterodimers to dissociate more readily or they result in a repositioning of the S2 site making it more accessible to metalloproteases. Some representative activating mutations found in human Notch1 are: VI 557E, F1593 S, L1594P, L1597H, R1599P, R1609S, 11617T, 11617N, V1677D, L1679P, L1681N, A1702P, 11719T. Additional mutations found in Notch1 of T-ALL are disclosed in Weng et al. (2004, Science 306, 269-271) and U.S. patent application Ser. No. 11/194,913, which are herein incorporated by reference.

In the immune system, the Notch pathway has been most studied in the area of lymphocyte development. Notch plays a critical role in thymocyte development where it promotes T versus B lymphocyte lineage commitment, determines T cell receptor usage and influences expression of CD4 and CD8 (Guidos, 2002, Semin. Immun. 14, 395-404). In mature T-cells, TGF-beta induced signaling and the Notch1 pathway seem to be integrated (Ostroukhavo et al., 2006, J. Clin. Inv. 116, 996-1004). In antigen induced tolerance, activation of the TGF-beta pathway triggers Notch activation on the target T-cell inhibiting cell activation.

Inhibition of the Notch1 activation, for instance by a specific gamma-secretase inhibitor, attenuates the immunosuppressive potential of antigen-induced tolerance (Ostroukhavo et al., 2006, J. Clin. Inv. 116, 996-1004). The Notch1 pathway has been suggested to be a common mechanism for maintenance of immune homeostasis in both naïve and antigen-provoked animals. Local activation of Notch1 at the site of inflammation (Lungs in asthma or joints in rheumatoid arthritis) may prove to be a novel approach to tame aberrant immune activation in chronic inflammatory disease states.

Thus, the molecules of the invention are useful for treating immune disorders. An “immune disorder” includes adult respiratory distress syndrome, arteriosclerosis, asthma, atherosclerosis, cholecystitis, cirrhosis, Crohn's disease, diabetes mellitus, emphysema, hypereosinophilia, inflammation, irritable bowel syndrome, multiple sclerosis, myasthenia gravis, myocardial or pericardial inflammation, osteoarthritis, osteoporosis, pancreatitis, rheumatoid arthritis, scleroderma, and colitis (see, e.g., US published application 2003/0175754).

“Inflammatory disorders” include diseases such as rheumatoid arthritis, Crohn's disease, mastocytosis, asthmas, multiple sclerosis, inflammatory bowel syndrome and allergic rhinitis (see, e.g., US published application 2006/0058340).

Studies have shown that Notch1 and Notch4 are elementary in vascular development. Appropriate levels of Notch signaling are critical for embryonic vascular development as both knock-out and activated mutants lead to embryonic death (Uyttendaele et al., 2001, Proc. Nat. Acad. Sci. 98, 5643-5648). The function of Notch in vascular homeostasis can also be drawn from the human neurovascular disorder CADASIL (Cerebral Autosomal Dominant Arteiopathy with Subcortical Infarcts and Leukoencephalopathy). The disorder is a late-onset autosomal dominant disorder, characterized by migraines and recurrent strokes, that results from one of a number of missense mutations in human Notch3 (Viitanen et al., 2000, Ann. N.Y. Acad. Sci. 903, 273-284). The disorder further manifests itself by disorganization and destruction of the vascular smooth muscle cells surrounding the cerebral arteries and arterioles and correlates with the expression of Notch 3 in vascular smooth muscle cells. Studies. of the Alagille Syndrome (AGS) have implicated Notch signaling in organ-specific angiogenesis. AGS syndrome is characterized by abnormalities of the liver, heart, kidney and other organs. About 60-70% of the AGS cases have shown mutations in Jagged1, one of the Serrate class of ligands in mammals that binds to Notch2. Some of these mutations render Jagged 1 incapable of activating the Notch2 receptor, implicating a role for Notch signaling in organ-specific angiogenesis (Morrisette et al., 2001, Hum. Mol. Genet. 10, 405-413).

Thus, the invention is also useful for treating disorders of the vasculature. Disorders of the vasculature, also termed “vascular disorders”, in addition to CADASIL and stroke, that can be treated or prevented according to the methods of the invention include atheroma, tumor angiogenesis, wound healing, diabetic retinopathy, hemangioma, psoriasis, and restenosis, e.g., restenosis resulting from balloon angioplasty (see e.g. US published application 2003/0180784).

By angiogenesis herein is meant a disease state which is marked by either an excess or a deficit of blood vessel development. Angiogenesis disorders associated with increased angiogenesis include, but are not limited to, cancer and proliferative diabetic retinopathy. Pathological states for which it may be desirable to increase angiogenesis include stroke, heart disease, infertility, ulcers, wound healing, ischemia, and scleroderma. Solid tumors typically require angiogenesis to support or sustain growth, e.g., breast, colon, lung, brain, bladder, and prostate tumors. Other angiogenesis disorders include, e.g., arthritis, inflammatory bowel disease, diabetes retinopathy, macular degeneration, atherosclerosis, and psoriasis (See e.g. U.S. published application 2004/0033495).

The compounds of the invention are also useful in the treatment of kidney disease. The role of Notch signaling in kidney development was shown by studies in mice with gamma-secretase inhibitors (Cheng et al., 2003, 5031-5042). Inactivating gamma-secretase inhibits Notch receptor signaling because the receptor can no longer be cleaved. The studies showed an absolute requirement for gamma-secretase in the transition from primitive epithelia to proximal nephron. Later studied showed that gamma-secretase activity, probably through activation of Notch is essential in kidney development for forming of the proximal tubule and podocyte fates when S-shaped bodies are forming (Cheng et al., 2005, Kidney Int. 68, 1951-1952).

The role of Notch in T cell development is well established (e.g. Aster, 2005, Int. J. Hematol. 82, 295-301). Notch1 can drive pluripotent bone marrow cells towards cell fate and expand the pool of immature T cell progenitors which are exceptionally susceptible to transformation by Notch (Pui et al., 1999, Immunity 11:299-308). Self-renewal of hematopoietic stem cells is regulated by inhibition of differentiation and induction of proliferation. Notch signaling has been revealed as a key factor in inhibiting differentiation (Duncan et al., 2005. Nat. Immunol. 6, 234). Inhibition of Notch led to accelerated differentiation of HCSs in vitro and depletion of HCSs in vivo.

The importance of notch in stem cell renewal has also been seen in other stem and precursor cells. Notch signaling has been shown to act on mammary stem cells and promote self-renewal in early progenitor cells resulting in increased proliferation. Notch signaling is also able to act on multipotent progenitor cells, facilitating myoepithelial lineage-specific commitment and proliferation. The effects were inhibited by addition of a gamma-secretase inhibitor (Dortu et al., 2004, Breast Canc. Res. 6, R605-615). Many neuronal precursor cells (NPC) undergo apoptosis during the development of the mammalian brain. The survival of mouse embryonic NPCs in vitro was increased by culture at high cell density and attributable to activation of Notch signaling (Oishi et al., 2004, Dev. Biol. 172-184).

An increase in Notch signaling results in an increase cell survival in a number of cell types. The ability to increase Notch signaling may therefore be a valuable tool in the transformation and proliferation of cells. The LNR-HD domain of Notch offers a potential element to increase Notch signaling. The LNR-HD domain may be ‘locked’ in its ‘on’ form, which allows for cleavage by metalloproteases at S2, resulting in an increase in Notch signaling and cell proliferation. Compounds that ‘lock’ the LNR-HD receptor in its on form may therefore be potentially useful agents in the maintenance of stem cells and other non-transformed human cell lines.

The compounds of the invention are useful in effective amounts. The term effective amount refers to the amount necessary or sufficient to realize a desired biologic effect. Combined with the teachings provided herein, by choosing among the various active compounds and weighing factors such as potency, relative bioavailability, patient body weight, severity of adverse side-effects and preferred mode of administration, an effective prophylactic or therapeutic treatment regimen can be planned which does not cause substantial toxicity and yet is effective to treat the particular subject. The effective amount for any particular application can vary depending on such factors as the disease or condition being treated, the particular LNR-HD domain or antibody being administered, the size of the subject, or the severity of the disease or condition. One of ordinary skill in the art can empirically determine the effective amount of a particular LNR-HD domain or antibody and/or other therapeutic agent without necessitating undue experimentation. It is preferred generally that a maximum dose be used, that is, the highest safe dose according to some medical judgment. Multiple doses per day may be contemplated to achieve appropriate systemic levels of compounds. Appropriate system levels can be determined by, for example, measurement of the patient's peak or sustained plasma level of the drug. “Dose” and “dosage” are used interchangeably herein.

Generally, daily oral doses of active compounds will be from about 0.01 milligrams/kg per day to 1000 milligrams/kg per day. It is expected that oral doses in the range of 0.5 to 50 milligrams/kg, in one or several administrations per day, will yield the desired results. Dosage may be adjusted appropriately to achieve desired drug levels, local or systemic, depending upon the mode of administration. For example, it is expected that intravenous administration would be from an order to several orders of magnitude lower dose per day. In the event that the response in a subject is insufficient at such doses, even higher doses (or effective higher doses by a different, more localized delivery route) may be employed to the extent that patient tolerance permits. Multiple doses per day are contemplated to achieve appropriate systemic levels of compounds.

A number of nucleic acid vector constructs of Notch receptors domains have been expressed in mammalian cells to elucidate the function of the various domains (Sanchez-Irizarry et al., 2004, Mol. Cell. Biol. 24, 9265-9273). This invention describes in detail the expression of the LNR-HD domains in its native structure by deleting predicted disordered sequences (See Table 1 and Table 2). The deleted sequences are not essential for the overall structure of the domains as the core structural elements of the domains were not altered. The native domain was further reconstituted by a correct refolding of the many cysteine groups within the LNR domains after bacterial expression. The correct pairing of the cysteine bonds was achieved by carefully monitoring and managing the redox potential during the refolding reaction after expression and was checked by MALDI-TOF and ESI-MS analysis. Incorrectly folded proteins were separated from the correctly folded proteins by anion exchange chromatography rather than by HPLC separation (A more conventional technique).

The invention also encompasses the use of LNR-HD nucleic acids. As used herein, the term “nucleic acid” is used to mean one or more nucleotides, i.e. a molecule comprising a sugar (e.g. ribose or deoxyribose) linked to a phosphate group and to an exchangeable organic base, which may be a substituted pyrimidine (e.g. cytosine (C), thymidine (T) or uracil (U)) or a substituted purine (e.g. adenine (A) or guanine (G)). The term “nucleic acid” also includes “polynucleotides” or “oligonucleotides,” as those terms are ordinarily used in the art, i.e., polymers of nucleotides, where oligonucleotides are generally shorter in length than polynucleotides. A sequence of nucleotides bonded together, i.e., within a polynucleotide or an oligonucleotide can be referred to as a “nucleotide sequence.” The term “nucleic acid” also includes nucleosides and polynucleosides (i.e. a nucleotide/polynucleotide without the phosphate). Purines and pyrimidines include, but are not limited to, natural nucleosides. In some embodiments the nucleic acid is isolated.

In one embodiment nucleic acid encodes the sequence of the LNR-HD domain of Notch1 or Notch2, which can be used, for example, to express native or modified LNR-HD in cells. In another embodiment nucleic acid includes siRNA, antisense DNA, antisense RNA and aptamers, which may be used, for example, to reduce expression of LNR-HD in cells.

A “small interfering RNA” or “siRNA,” as used herein, refers to a RNA molecule derived from the successive cleavage of long double-stranded RNA (dsRNA) within a cell to produce an RNA molecule generally have a length of between 15 and 30 nucleotides, and more often between 20 and 25 nucleotides. siRNAs direct the destruction of corresponding mRNA targets during RNA interference in animals, and during other RNA-silencing phenomena, including posttranscriptional gene silencing of plants and quelling of Neurospora.

The methods for design of the RNAs that mediate RNAi and the methods for transfection of the RNAs into cells and animals is well known in the art and are readily commercially available (Verma N. K. et al, 2004, J. Clin. Pharm. Ther., 28, 395-404; Mello C. C. et al. 2004, Nature, 431, 338-42; Dykxhoom D. M. et al., 2003, Nat. Rev. Mol. Cell. Biol. 4, 457-67; Proligo (Hamburg, Germany), Dharmacon Research (Lafayette, Colo., USA), Pierce Chemical (part of Perbio Science, Rockford, Ill., USA), Glen Research (Sterling, Va., USA), ChemGenes (Ashland, Mass., USA), and Cruachem (Glasgow, UK)). The RNAs are preferably chemically synthesized using appropriately protected ribonucleoside phosphoramidites and a conventional DNA/RNA synthesizer. Most conveniently, siRNAs are obtained from commercial RNA oligo synthesis suppliers listed herein. In general, RNAs are not too difficult to synthesize and are readily provided in a quality suitable for RNAi. A typical 0.2 lμmol-scale RNA synthesis provides about 1 milligram of RNA, which is sufficient for 1000 transfection experiments using a 24-well tissue culture plate format.

The LNR-HD domain cDNA specific siRNA is designed preferably by selecting a sequence that is not within 50-100 bp of the start codon and the termination codon, avoids intron regions, avoids stretches of 4 or more bases such as AAAA, CCCC, avoids regions with GC content <30% or >60%, avoids repeats and low complex sequence, and it avoids single nucleotide polymorphism sites. The LNR-HD domain siRNA may be designed by a search for a 23-nt sequence motif AA(N19). If no suitable sequence is found, then a 23-nt sequence motif NA(N21) may be used with conversion of the 3′ end of the sense siRNA to TT. Alternatively, the LNR-HD domain siRNA can be designed by a search for NAR(N17)YNN. The target sequence may have a GC content of around 50%. The siRNA targeted sequence may be further evaluated using a BLAST homology search to avoid off target effects on other genes or sequences. Negative controls are designed by scrambling targeted siRNA sequences. The control RNA preferably has the same length and nucleotide composition as the siRNA but has at least 4-5 bases mismatched to the siRNA. The RNA molecules of the present invention can comprise a 3′ hydroxyl group. The RNA molecules can be single-stranded or double stranded; such molecules can be blunt ended or comprise overhanging ends (e.g., 5′, 3′) from about 1 to about 6 nucleotides in length (e.g., pyrimidine nucleotides, purine nucleotides). In order to further enhance the stability of the RNA of the present invention, the 3′ overhangs can be stabilized against degradation. The RNA can be stabilized by including purine nucleotides, such as adenosine or guanosine nucleotides. Alternatively, substitution of pyrimidine nucleotides by modified analogues, e.g., substitution of uridine 2 nucleotide 3′ overhangs by 2′-deoxythymidine is tolerated and does not affect the efficiency of RNAi. The absence of a 2′ hydroxyl significantly enhances the nuclease resistance of the overhang in tissue culture medium.

The RNA molecules used in the methods of the present invention can be obtained using a number of techniques known to those of skill in the art. For example, the RNA can be chemically synthesized or recombinantly produced using methods known in the art. Such methods are described in U.S. Published Patent Application Nos. US 2002-0086356A1 and US 2003-0206884A1 that are hereby incorporated by reference in their entirety.

The methods described herein are used to identify or obtain RNA molecules that are useful as sequence-specific mediators of LNR-HD domain mRNA degradation and, thus, for inhibiting LNR-HD domain activity. Expression of LNR-HD domain can be inhibited in humans in order to prevent the protein from being translated.

The RNA molecules may also be isolated using a number of techniques known to those of skill in the art. For example, gel electrophoresis can be used to separate RNAs from the combination, gel slices comprising the RNA sequences removed and RNAs eluted from the gel slices. Alternatively, non-denaturing methods, such as non-denaturing column chromatography, can be used to isolate the RNA produced. In addition, chromatography (e.g., size exclusion chromatography), glycerol gradient centrifugation, affinity purification with antibody can be used to isolate RNAs.

Any RNA can be used in the methods of the present invention, provided that it has sufficient homology to the LNR-HD domain gene to mediate RNAi. The RNA for use in the present invention can correspond to the entire LNR-HD domain gene or a portion thereof. There is no upper limit on the length of the RNA that can be used. For example, the RNA can range from about 21 base pairs (bp) of the gene to the full length of the gene or more. In one embodiment, the RNA used in the methods of the present invention is about 1000 bp in length. In another embodiment, the RNA is about 500 bp in length. In yet another embodiment, the RNA is about 22 bp in length. In certain embodiments the preferred length of the RNA of the invention is 21 to 23 nucleotides.

Antisense molecules can be modified or unmodified RNA, DNA, or mixed polymer oligonucleotides and primarily function by specifically binding to matching sequences resulting in modulation of peptide synthesis (Wu-Pong, November 1994, BioPharm, 20-33). The antisense oligonucleotide binds to target RNA by Watson Crick base-pairing and blocks gene expression by preventing ribosomal translation of the bound sequences either by steric blocking or by activating RNase H enzyme. Antisense molecules may also alter protein synthesis by interfering with RNA processing or transport from the nucleus into the cytoplasm (Mukhopadhyay & Roth, 1996, Crit. Rev. in Oncogenesis 7, 151-190).

As used herein, the term “antisense oligonucleotide” or “antisense” describes an oligonucleotide that is an oligoribonucleotide, oligodeoxyribonucleotide, modified oligoribonucleotide, or modified oligodeoxyribonucleotide which hybridizes under physiological conditions to DNA comprising a particular gene or to an mRNA transcript of that gene and, thereby, inhibits the transcription of that gene and/or the translation of that mRNA. The antisense molecules are designed so as to interfere with transcription or translation of a target gene upon hybridization with the target gene or transcript. Antisense oligonucleotides that selectively bind to a nucleic acid molecule encoding a LNR-HD domain are particularly preferred. Those skilled in the art will recognize that the exact length of the antisense oligonucleotide and its degree of complementarity with its target will depend upon the specific target selected, including the sequence of the target and the particular bases which comprise that sequence.

It is preferred that the antisense oligonucleotide be constructed and arranged so as to bind selectively with the target under physiological conditions, i.e., to hybridize substantially more to the target sequence than to any other sequence in the target cell under physiological conditions. Based upon the nucleotide sequences of nucleic acid molecules encoding LNR-HD domain, or upon allelic or homologous genomic and/or cDNA sequences, one of skill in the art can easily choose and synthesize any of a number of appropriate antisense molecules for use in accordance with the present invention. In order to be sufficiently selective and potent for inhibition, such antisense oligonucleotides should comprise at least about 10 and, more preferably, at least about 15 consecutive bases which are complementary to the target, although in certain cases modified oligonucleotides as short as 7 bases in length have been used successfully as antisense oligonucleotides. See Wagner et al., 1995, Nat. Med. 1, 1116-1118. Most preferably, the antisense oligonucleotides comprise a complementary sequence of 20-30 bases. Although oligonucleotides may be chosen which are antisense to any region of the gene or mRNA transcripts, in preferred embodiments the antisense oligonucleotides correspond to N-terminal or 5′ upstream sites such as translation initiation, transcription initiation or promoter sites. In addition, 3′-untranslated regions may be targeted by antisense oligonucleotides. Targeting to mRNA splicing sites has also been used in the art but may be less preferred if alternative mRNA splicing occurs. In addition, the antisense is targeted, preferably, to sites in which mRNA secondary structure is not expected (see, e.g., Sainio et al., 1994, Cell. Mol. Neurobiol. 14, 439-457) and at which proteins are not expected to bind.

In one set of embodiments, the antisense oligonucleotides of the invention may be composed of “natural” deoxyribonucleotides, ribonucleotides, or any combination thereof. That is, the 5′ end of one native nucleotide and the 3′ end of another native nucleotide may be covalently linked, as in natural systems, via a phosphodiester internucleoside linkage. These oligonucleotides may be prepared by art recognized methods which may be carried out manually or by an automated synthesizer. They also may be produced recombinantly by vectors.

In preferred embodiments, however, the antisense oligonucleotides of the invention also may include “modified” oligonucleotides. That is, the oligonucleotides may be modified in a number of ways which do not prevent them from hybridizing to their target but which enhance their stability or targeting or which otherwise enhance their therapeutic effectiveness.

The term “modified oligonucleotide” as used herein describes an oligonucleotide in which (1) at least two of its nucleotides are covalently linked via a synthetic internucleoside linkage (i.e., a linkage other than a phosphodiester linkage between the 5′ end of one nucleotide and the 3′ end of another nucleotide) and/or (2) a chemical group not normally associated with nucleic acid molecules has been covalently attached to the oligonucleotide. Preferred synthetic internucleoside linkages are phosphorothioates, alkylphosphonates, phosphorodithioates, phosphate esters, alkylphosphonothioates, phosphoramidates, carbamates, carbonates, phosphate triesters, acetamidates, carboxymethyl esters and peptides.

The term “modified oligonucleotide” also encompasses oligonucleotides with a covalently modified base and/or sugar. For example, modified oligonucleotides include oligonucleotides having backbone sugars which are covalently attached to low molecular weight organic groups other than a hydroxyl group at the 3′ position and other than a phosphate group at the 5′ position. Thus modified oligonucleotides may include a 2′-O-alkylated ribose group. In addition, modified oligonucleotides may include sugars such as arabinose instead of ribose.

The present invention, thus, contemplates pharmaceutical preparations containing modified antisense molecules that are complementary to and hybridizable with, under physiological conditions, nucleic acid molecules encoding a LNR-HD domain, together with pharmaceutically acceptable carriers. Antisense oligonucleotides may be administered as part of a pharmaceutical composition. In this latter embodiment, it may be preferable that a slow intravenous administration be used. Such a pharmaceutical composition may include the antisense oligonucleotides in combination with any standard physiologically and/or pharmaceutically acceptable carriers which are known in the art. The compositions should be sterile and contain a therapeutically effective amount of the antisense oligonucleotides in a unit of weight or volume suitable for administration to a subject.

By “aptamer” or “nucleic acid aptamer” as used herein is meant a nucleic acid molecule that binds specifically to a target molecule wherein the nucleic acid molecule has sequence that comprises a sequence recognized by the target molecule in its natural setting. Alternately, an aptamer can be a nucleic acid molecule that binds to a target molecule where the target molecule does not naturally bind to a nucleic acid. The target molecule can be any molecule of interest. For example, the aptamer can be used to bind to a ligand-binding domain of a protein, thereby preventing interaction of the naturally occurring ligand with the protein. This is a non-limiting example and those in the art will recognize that other embodiments can be readily generated using techniques generally known in the art.

Thus, one of ordinary skill in the art, in light of the present disclosure, is enabled to produce the Notch domains by standard technology, including recombinant technology, direct synthesis, mutagenesis, etc. For instance, using recombinant technology one may substitute appropriate codons to produce the desired amino acid substitutions by standard site-directed mutagenesis techniques. Obviously, one may also use any sequence which differs only due to the degeneracy of the genetic code as the starting point for site directed mutagenesis. The mutated nucleic acid sequence may then be ligated into an appropriate expression vector and expressed in a host such as E. coli. The resultant modified Notch domains may then be purified by techniques well known in the art, including those disclosed below in the Examples. Preferably the Notch domains are substantially pure. As used herein, the term “substantially pure” means that the proteins are essentially free of other substances to an extent practical and appropriate for their intended use. In particular, the proteins are sufficiently pure and are sufficiently free from other biological constituents of their host cells so as to be useful in, for example, protein sequencing, or producing pharmaceutical preparations.

In another set of embodiments an isolated nucleic acid encoding the modified Notch domains of the invention is provided. As used herein with respect to nucleic acids, the term “isolated” means: (i) amplified in vitro by, for example, polymerase chain reaction (PCR); (ii) recombinantly produced by cloning; (iii) purified, as by cleavage and gel separation; or (iv) synthesized by, for example, chemical synthesis. An isolated nucleic acid is one which is readily manipulable by recombinant DNA techniques well known in the art. Thus, a nucleotide sequence contained in a vector in which 5′ and 3′ restriction sites are known or for which polymerase chain reaction (PCR) primer sequences have been disclosed is considered isolated but a nucleic acid sequence existing in its native state in its natural host is not. An isolated nucleic acid may be substantially purified, but need not be. For example, a nucleic acid that is isolated within a cloning or expression vector is not pure in that it may comprise only a tiny percentage of the material in the cell in which it resides. Such a nucleic acid is isolated, however, as the term is used herein because it is readily manipulable by standard techniques known to those of ordinary skill in the art.

As used herein, a coding sequence and regulatory sequences are said to be “operably joined” when they are covalently linked in such a way as to place the expression or transcription of the coding sequence under the influence or control of the regulatory sequences. In order that the coding sequences be translated into a functional protein the coding sequences are operably joined to regulatory sequences. Two DNA sequences are said to be operably joined if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operably joined to a coding sequence if the promoter region were capable of effecting transcription of that DNA sequence such that the resulting transcript might be translated into the desired protein or polypeptide.

The precise nature of the regulatory sequences needed for gene expression may vary between species or cell types, but shall in general include, as necessary, 5′ non-transcribing and 5′ non-translating sequences involved with initiation of transcription and translation respectively, such as a TATA box, capping sequence, CAAT sequence, and the like. Especially, such 5′ non-transcribing regulatory sequences will include a promoter region which includes a promoter sequence for transcriptional control of the operably joined gene. Promoters may be constitutive or inducible. Regulatory sequences may also include enhancer sequences or upstream activator sequences, as desired.

As used herein, a “vector” may be any of a number of nucleic acids into which a desired sequence may be inserted by restriction and ligation for transport between different genetic environments or for expression in a host cell. Vectors are typically composed of DNA although RNA vectors are also available. Vectors include, but are not limited to, plasmids and phagemids. A cloning vector is one which is able to replicate in a host cell, and which is further characterized by one or more endonuclease restriction sites at which the vector may be cut in a determinable fashion and into which a desired DNA sequence may be ligated such that the new recombinant vector retains its ability to replicate in the host cell. In the case of plasmids, replication of the desired sequence may occur many times as the plasmid increases in copy number within the host bacterium, or just a single time per host as the host reproduces by mitosis. In the case of phage, replication may occur actively during a lytic phase or passively during a lysogenic phase. An expression vector is one into which a desired DNA sequence may be inserted by restriction and ligation such that it is operably joined to regulatory sequences and may be expressed as an RNA transcript. Vectors may further contain one or more marker sequences suitable for use in the identification of cells which have or have not been transformed or transfected with the vector. Markers include, for example, genes encoding proteins which increase or decrease either resistance or sensitivity to antibiotics or other compounds, genes which encode enzymes whose activities are detectable by standard assays known in the art (e.g., β-galactosidase or alkaline phosphatase), and genes which visibly affect the phenotype of transformed or transfected cells, hosts, colonies or plaques. Preferred vectors are those capable of autonomous replication and expression of the structural gene products present in the DNA segments to which they are operably joined.

As used herein, the term “stringent conditions” refers to parameters known to those skilled in the art. One example of stringent conditions is hybridization at 65° C. in hybridization buffer (3.5×SSC, 0.02% Ficoll, 0.02% polyvinyl pyrolidone, 0.02% bovine serum albumin (BSA), 25 mM NaH₂PO₄ (pH7), 0.5% SDS, 2 mM EDTA). SSC is 0.15M sodium chloride/0.15M sodium citrate, pH7; SDS is sodium dodecylsulphate; and EDTA is ethylene diamine tetra acetic acid. There are other conditions, reagents, and so forth which can be used, which result in the same degree of stringency. A skilled artisan will be familiar with such conditions, and thus they are not given here. The skilled artisan also is familiar with the methodology for screening cells for expression of such molecules, which then are routinely isolated, followed by isolation of the pertinent nucleic acid. Thus, homologs and alleles of the LNR-HD domain of the invention, as well as nucleic acids encoding the same, may be obtained routinely, and the invention is not intended to be limited to the specific sequences disclosed.

For prokaryotic systems, plasmid vectors that contain replication sites and control sequences derived from a species compatible with the host may be used. Examples of suitable plasmid vectors include pBR322, pUC18, pUC19 and the like; suitable phage or bacteriophage vectors include λgt10, λgt11 and the like; and suitable virus vectors include pMAM-neo, pKRC and the like. Preferably, the selected vector of the present invention has the capacity to autonomously replicate in the selected host cell. Useful prokaryotic hosts include bacteria such as E. coli, Flavobacterium heparinum, Bacillus, Streptomyces, Pseudomonas, Salmonella, Serratia, and the like.

To express the LNR-HD domain of the invention in a prokaryotic cell, it is necessary to operably join the nucleic acid sequences of the monomers and the linker to a functional prokaryotic promoter. Such promoter may be either constitutive or, more preferably, regulatable (i.e., inducible or derepressible). Examples of constitutive promoters include the int promoter of bacteriophage %, the bla promoter of the β-lactamase gene sequence of pBR322, and the CAT promoter of the chloramphenicol acetyl transferase gene sequence of pPR325, and the like. Examples of inducible prokaryotic promoters include the major right and left promoters of bacteriophage λ (P_{L} and P_{R}), the trp, recA, lacZ, lacI, and gal promoters of E. coli the α-amylase (Ulmanen et al. 1985, J. Bacteriol. 162, 176-182) and the ζ-28-specific promoters of B. subtilis (Gilman et al. 1984 Gene sequence 32, 11-20), the promoters of the bacteriophages of Bacillus (Gryczan, 1982, in: The Molecular Biology of the Bacilli, Academic Press, Inc., NY), and Streptomyces promoters (Ward et al. 1986, Mol. Gen. Genet. 203, 468-478).

Prokaryotic promoters are reviewed by Glick (1987, J. Ind. Microbiol. 1, 277-282); Cenatiempo (1986, Biochimie 68, 505-516); and Gottesman (1984, Ann. Rev. Genet. 18, 415-442).

Proper expression in a prokaryotic cell also requires the presence of a ribosome binding site upstream of the encoding sequence. Such ribosome binding sites are disclosed, for example, by Gold et al. (1981, Ann. Rev. Microbiol. 35, 365-404).

Because prokaryotic cells will not produce the LNR-HD domain of the invention with normal eukaryotic glycosylation, expression of the LNR-HD domain of the invention by eukaryotic hosts is possible when glycosylation is desired. Preferred eukaryotic hosts include, for example, yeast, fungi, insect cells, and mammalian cells, either in vivo or in tissue culture. Mammalian cells which may be useful as hosts include HeLa cells, cells of fibroblast origin such as VERO or CHO-K1, or cells of lymphoid origin, such as the hybridoma SP2/0-AG14 or the myeloma P3x63Sg8, and their derivatives. Preferred mammalian host cells include SP2/0 and J558L, as well as neuroblastoma cell lines such as IMR 332 that may provide better capacities for correct post-translational processing. Embryonic cells and mature cells of a transplantable organ also are useful according to some aspects of the invention.

In addition, plant cells are also available as hosts, and control sequences compatible with plant cells are available, such as the nopaline synthase promoter and polyadenylation signal sequences.

Another preferred host is an insect cell, for example in Drosophila larvae. When using insect cells as hosts, the Drosophila alcohol dehydrogenase promoter can be used (Rubin, 1988, Science 240, 1453-1459). Alternatively, baculovirus vectors can be engineered to express large amounts of the LNR-HD domain of the invention in insects cells (Jasny, 1987, Science 238, 1653; Miller et al., 1986 in: Genetic Engineering, Setlow, J. K., et al., eds., Plenum, Vol. 8, pp. 277-297).

Any of a series of yeast gene sequence expression systems which incorporate promoter and termination elements from the genes coding for glycolytic enzymes and which are produced in large quantities when the yeast are grown in media rich in glucose may also be utilized. Known glycolytic gene sequences can also provide very efficient transcriptional control signals. Yeast provide substantial advantages in that they can also carry out post-translational peptide modifications. A number of recombinant DNA strategies exist which utilize strong promoter sequences and high copy number plasmids which can be utilized for production of the desired proteins in yeast. Yeast recognize leader sequences on cloned mammalian gene sequence products and secrete peptides bearing leader sequences (i.e., pre-peptides).

A wide variety of transcriptional and translational regulatory sequences may be employed, depending upon the nature of the host. The transcriptional and translational regulatory signals may be derived from viral sources, such as adenovirus, bovine papilloma virus, simian virus, or the like, where the regulatory signals are associated with a particular gene sequence which has a high level of expression. Alternatively, promoters from mammalian expression products, such as actin, collagen, myosin, and the like, may be employed. Transcriptional initiation regulatory signals may be selected which allow for repression or activation, so that expression of the gene sequences can be modulated. Of interest are regulatory signals which are temperature-sensitive so that by varying the temperature, expression can be repressed or initiated, or which are subject to chemical (such as metabolite) regulation.

As discussed above, expression of the LNR-HD domain of the invention in eukaryotic hosts requires the use of eukaryotic regulatory regions. Such regions will, in general, include a promoter region sufficient to direct the initiation of RNA synthesis. Preferred eukaryotic promoters include, for example, the promoter of the mouse metallothionein I gene sequence (Hamer et al. 1982, J. Mol. Appl. Gen. 1, 273-288); the TK promoter of Herpes virus (McKnight, 1982 Cell 31, 355-365); the SV40 early promoter (Benoist et al. 1981 Nature (London) 290, 304-310); the yeast gal4 gene sequence promoter (Johnston et al., 1982, Proc. Natl. Acad. Sci. (USA) 79, 6971-6975; Silver et al., 1984, Proc. Natl. Acad. Sci. (USA) 81, 5951-5955).

As is widely known, translation of eukaryotic mRNA is initiated at the codon which encodes the first methionine. For this reason, it is preferable to ensure that the linkage between a eukaryotic promoter and the DNA sequences which encode the LNR-HD domain of the invention does not contain any intervening codons which are capable of encoding a methionine (i.e., AUG). The presence of such codons results either in the formation of a fusion protein (if the AUG codon is in the same reading frame as the LNR-HD domain coding sequence) or a frame-shift mutation (if the AUG codon is not in the same reading frame as the LNR-HD domain coding sequence).

In one embodiment, a vector is employed which is capable of integrating the desired gene sequences into the host cell chromosome. Cells which have stably integrated the introduced DNA into their chromosomes can be selected by also introducing one or more markers which allow for selection of host cells which contain the expression vector. The marker may, for example, provide for prototrophy to an auxotrophic host or may confer biocide resistance to, e.g., antibiotics, heavy metals, or the like. The selectable marker gene sequence can either be directly linked to the DNA gene sequences to be expressed or introduced into the same cell by co-transfection. Additional elements may also be needed for optimal synthesis of the LNR-HD domain mRNA. These elements may include splice signals, as well as transcription promoters, enhancers, and termination signals. cDNA expression vectors incorporating such elements include those described by Okayama (1983, Molec. Cell. Biol. 3, 280).

In a preferred embodiment, the introduced sequence will be incorporated into a plasmid or viral vector capable of autonomous replication in the recipient host. Any of a wide variety of vectors may be employed for this purpose. Factors of importance in selecting a particular plasmid or viral vector include the following: the ease with which recipient cells that contain the vector may be recognized and selected from those recipient cells which do not contain the vector, the number of copies of the vector which are desired in a particular host and whether it is desirable to be able to “shuttle” the vector between host cells of different species. Preferred prokaryotic vectors include plasmids such as those capable of replication in E. coli (such as, for example, pBR322, ColE1, pSC101, pACYC 184, and πVX. Such plasmids are, for example, disclosed by Sambrook, et al. (1989, Molecular Cloning: A Laboratory Manual, second edition, edited by Sambrook, Fritsch, & Maniatis, Cold Spring Harbor Laboratory). Bacillus plasmids include pC194, pC221, pT127 and the like. Such plasmids are disclosed by Gryczan (1982, in: The Molecular Biology of the Bacilli, Academic Press, NY, pp. 307-329). Suitable Streptomyces plasmids include pIJ101 (Kendall et al. 1987, J. Bacteriol. 169, 4177-4183), and streptomyces bacteriophages such as φC31 (1986, Chater et al., In: Sixth International Symposium on Actinomycetales Biology, Akademiai Kaido, Budapest, Hungary, pp. 45-54). Pseudomonas plasmids are reviewed by John et al. (1986, Rev. Infect. Dis. 8, 693-704), and Izaki (1978, Jpn. J. Bacteriol. 33, 729-742).

Preferred eukaryotic plasmids include, for example, BPV, EBV, SV40, 2-micron circle, and the like, or their derivatives. Such plasmids are well known in the art (1982, Botstein et al., Miami Wntr. Symp. 19, 265-274); Broach, 1981, in: The Molecular Biology of the Yeast Saccharomyces: Life Cycle and Inheritance, Cold Spring Harbor Laboratory, Cold Spring Harbor, N.Y., p. 445-470; Broach, 1982, Cell 28:203-204; Bollon et al. 1980, J. Clin. Hematol. Oncol. 10:39-48; Maniatis, 1980, in: Cell Biology: A Comprehensive Treatise, Vol. 3, Gene Sequence Expression, Academic Press, NY, pp. 563-608). Other preferred eukaryotic vectors are viral vectors. For example, and not by way of limitation, the pox virus, herpes virus, adenovirus and various retroviruses may be employed. The viral vectors may include either DNA or RNA viruses to cause expression of the insert DNA or insert RNA. Additionally, DNA or RNA encoding the LNR-HD domain polypeptides may be directly injected into cells or may be impelled through cell membranes after being adhered to microparticles.

Once the vector or DNA sequence containing the construct(s) has been prepared for expression, the DNA construct(s) may be introduced into an appropriate host cell by any of a variety of suitable means, i.e., transformation, transfection, conjugation, protoplast fusion, electroporation, calcium phosphate-precipitation, direct microinjection, and the like. After the introduction of the vector, recipient cells are grown in a selective medium, which selects for the growth of vector-containing cells. Expression of the cloned gene sequence(s) results in the production of the LNR-HD domain. This can take place in the transformed cells as such, or following the induction of these cells to differentiate (for example, by administration of bromodeoxyuracil to neuroblastoma cells or the like).

The formulations of the invention are administered in pharmaceutically acceptable solutions, which may routinely contain pharmaceutically acceptable concentrations of salt, buffering agents, preservatives, compatible carriers, adjuvants, and optionally other therapeutic ingredients.

For use in therapy, an effective amount of the LNR-HD domain peptide or nucleic acid or antibody or fragment thereof (collectively referred to herein for purposes of brevity as LNR-HD domain) can be administered to a subject by any mode that delivers the LNR-HD domain to the desired surface. Administering the pharmaceutical composition of the present invention may be accomplished by any means known to the skilled artisan. Preferred routes of administration include but are not limited to oral, parenteral, intramuscular, intranasal, sublingual, intratracheal, inhalation, ocular, vaginal, and rectal.

For oral administration, the compounds (i.e., LNR-HD domains, and other therapeutic agents) can be formulated readily by combining the active compound(s) with pharmaceutically acceptable carriers well known in the art. Such carriers enable the compounds of the invention to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally the oral formulations may also be formulated in saline or buffers, i.e. EDTA for neutralizing internal acid conditions or may be administered without any carriers.

Also specifically contemplated are oral dosage forms of the above component or components. The component or components may be chemically modified so that oral delivery of the derivative is efficacious. Generally, the chemical modification contemplated is the attachment of at least one moiety to the component molecule itself, where said moiety permits (a) inhibition of proteolysis; and (b) uptake into the blood stream from the stomach or intestine. Also desired is the increase in overall stability of the component or components and increase in circulation time in the body. Examples of such moieties include: polyethylene glycol, copolymers of ethylene glycol and propylene glycol, carboxymethyl cellulose, dextran, polyvinyl alcohol, polyvinyl pyrrolidone and polyproline. Abuchowski and Davis, 1981, “Soluble Polymer-Enzyme Adducts” In: Enzymes as Drugs, Hocenberg and Roberts, eds., Wiley-Interscience, New York, N.Y., pp. 367-383; Newmark, et al., 1982, J. Appl. Biochem. 4:185-189. Other polymers that could be used are poly-1,3-dioxolane and poly-1,3,6-tioxocane. Preferred for pharmaceutical usage, as indicated above, are polyethylene glycol moieties.

For the component (or derivative) the location of release may be the stomach, the small intestine (the duodenum, the jejunum, or the ileum), or the large intestine. One skilled in the art has available formulations which will not dissolve in the stomach, yet will release the material in the duodenum or elsewhere in the intestine. Preferably, the release will avoid the deleterious effects of the stomach environment, either by protection of the LNR-HD domain (or derivative) or by release of the biologically active material beyond the stomach environment, such as in the intestine.

To ensure full gastric resistance a coating impermeable to at least pH 5.0 is essential. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and Shellac. These coatings may be used as mixed films.

A coating or mixture of coatings can also be used on tablets, which are not intended for protection against the stomach. This can include sugar coatings, or coatings which make the tablet easier to swallow. Capsules may consist of a hard shell (such as gelatin) for delivery of dry therapeutic i.e. powder; for liquid forms, a soft gelatin shell may be used. The shell material of cachets could be thick starch or other edible paper. For pills, lozenges, molded tablets or tablet triturates, moist massing techniques can be used.

The therapeutic can be included in the formulation as fine multi-particulates in the form of granules or pellets of particle size about 1 mm. The formulation of the material for capsule administration could also be as a powder, lightly compressed plugs or even as tablets. The therapeutic could be prepared by compression.

Colorants and flavoring agents may all be included. For example, the LNR-HD domain (or derivative) may be formulated (such as by liposome or microsphere encapsulation) and then further contained within an edible product, such as a refrigerated beverage containing colorants and flavoring agents.

One may dilute or increase the volume of the therapeutic with an inert material. These diluents could include carbohydrates, especially mannitol, a-lactose, anhydrous lactose, cellulose, sucrose, modified dextrans and starch. Certain inorganic salts may be also be used as fillers including calcium triphosphate, magnesium carbonate and sodium chloride. Some commercially available diluents are Fast-Flo, Emdex, STA-Rx 1500, Emcompress and Avicell.

Disintegrants may be included in the formulation of the therapeutic into a solid dosage form. Materials used as disintegrates include but are not limited to starch, including the commercial disintegrant based on starch, Explotab. Sodium starch glycolate, Amberlite, sodium carboxymethylcellulose, ultramylopectin, sodium alginate, gelatin, orange peel, acid carboxymethyl cellulose, natural sponge and bentonite may all be used. Another form of the disintegrants are the insoluble cationic exchange resins. Powdered gums may be used as disintegrants and as binders and these can include powdered gums such as agar, Karaya or tragacanth. Alginic acid and its sodium salt are also useful as disintegrants.

Binders may be used to hold the therapeutic agent together to form a hard tablet and include materials from natural products such as acacia, tragacanth, starch and gelatin. Others include methyl cellulose (MC), ethyl cellulose (EC) and carboxymethyl cellulose (CMC). Polyvinyl pyrrolidone (PVP) and hydroxypropylmethyl cellulose (HPMC) could both be used in alcoholic solutions to granulate the therapeutic.

An anti-frictional agent may be included in the formulation of the therapeutic to prevent sticking during the formulation process. Lubricants may be used as a layer between the therapeutic and the die wall, and these can include but are not limited to; stearic acid including its magnesium and calcium salts, polytetrafluoroethylene (PTFE), liquid paraffin, vegetable oils and waxes. Soluble lubricants may also be used such as sodium lauryl sulfate, magnesium lauryl sulfate, polyethylene glycol of various molecular weights, Carbowax 4000 and 6000.

Glidants that might improve the flow properties of the drug during formulation and to aid rearrangement during compression might be added. The glidants may include starch, talc, pyrogenic silica and hydrated silicoaluminate.

To aid dissolution of the therapeutic into the aqueous environment a surfactant might be added as a wetting agent. Surfactants may include anionic detergents such as sodium lauryl sulfate, dioctyl sodium sulfosuccinate and dioctyl sodium sulfonate. Cationic detergents might be used and could include benzalkonium chloride or benzethomium chloride. The list of potential non-ionic detergents that could be included in the formulation as surfactants are lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose. These surfactants could be present in the formulation of the LNR-HD domain or derivative either alone or as a mixture in different ratios.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration.

For buccal administration, the compositions may take the form of tablets or lozenges formulated in conventional manner.

For administration by inhalation, the compounds for use according to the present invention may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

Also contemplated herein is pulmonary delivery of the LNR-HD domains (or derivatives thereof). The LNR-HD domain (or derivative) is delivered to the lungs of a mammal while inhaling and traverses across the lung epithelial lining to the blood stream. Other reports of inhaled molecules include Adjei et al., 1990, Pharmaceutical Research, 7:565-569; Adjei et al., 1990, International Journal of Pharmaceutics, 63:135-144 (leuprolide acetate); Braquet et al., 1989, Journal of Cardiovascular Pharmacology, 13(suppl. 5):143-146 (endothelin-1); Hubbard et al., 1989, Annals of Internal Medicine, Vol. III, pp. 206-212 (a1-antitrypsin); Smith et al., 1989, J. Clin. Invest. 84:1145-1146 (a-1-proteinase); Oswein et al., 1990, “Aerosolization of Proteins”, Proceedings of Symposium on Respiratory Drug Delivery II, Keystone, Colo., March, (recombinant human growth hormone); Debs et al., 1988, J. Immunol. 140:3482-3488 (interferon-g and tumor necrosis factor alpha) and Platz et al., U.S. Pat. No. 5,284,656 (granulocyte colony stimulating factor). A method and composition for pulmonary delivery of drugs for systemic effect is described in U.S. Pat. No. 5,451,569, issued Sep. 19, 1995 to Wong et al.

Contemplated for use in the practice of this invention are a wide range of mechanical devices designed for pulmonary delivery of therapeutic products, including but not limited to nebulizers, metered dose inhalers, and powder inhalers, all of which are familiar to those skilled in the art.

Some specific examples of commercially available devices suitable for the practice of this invention are the Ultravent nebulizer, manufactured by Mallinckrodt, Inc., St. Louis, Mo.; the Acorn II nebulizer, manufactured by Marquest Medical Products, Englewood, Colo.; the Ventolin metered dose inhaler, manufactured by Glaxo Inc., Research Triangle Park, N.C.; and the Spinhaler powder inhaler, manufactured by Fisons Corp., Bedford, Mass.

All such devices require the use of formulations suitable for the dispensing of LNR-HD domain (or derivative). Typically, each formulation is specific to the type of device employed and may involve the use of an appropriate propellant material, in addition to the usual diluents, adjuvants and/or carriers useful in therapy. Also, the use of liposomes, microcapsules or microspheres, inclusion complexes, or other types of carriers is contemplated. Chemically modified LNR-HD domain may also be prepared in different formulations depending on the type of chemical modification or the type of device employed.

Formulations suitable for use with a nebulizer, either jet or ultrasonic, will typically comprise LNR-HD domain (or derivative) dissolved in water at a concentration of about 0.1 to 25 mg of biologically active LNR-HD domain per mL of solution. The formulation may also include a buffer and a simple sugar (e.g., for LNR-HD domain stabilization and regulation of osmotic pressure). The nebulizer formulation may also contain a surfactant, to reduce or prevent surface induced aggregation of the LNR-HD domain caused by atomization of the solution in forming the aerosol.

Formulations for use with a metered-dose inhaler device will generally comprise a finely divided powder containing the LNR-HD domain (or derivative) suspended in a propellant with the aid of a surfactant. The propellant may be any conventional material employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1,1,2-tetrafluoroethane, or combinations thereof. Suitable surfactants include sorbitan trioleate and soya lecithin. Oleic acid may also be useful as a surfactant.

Formulations for dispensing from a powder inhaler device will comprise a finely divided dry powder containing LNR-HD domain (or derivative) and may also include a bulking agent, such as lactose, sorbitol, sucrose, or mannitol in amounts which facilitate dispersal of the powder from the device, e.g., 50 to 90% by weight of the formulation. The LNR-HD domain (or derivative) should most advantageously be prepared in particulate form with an average particle size of less than 10 mm (or microns), most preferably 0.5 to 5 mm, for most effective delivery to the distal lung.

Nasal delivery of a pharmaceutical composition of the present invention is also contemplated. Nasal delivery allows the passage of a pharmaceutical composition of the present invention to the blood stream directly after administering the therapeutic product to the nose, without the necessity for deposition of the product in the lung. Formulations for nasal delivery include those with dextran or cyclodextran.

For nasal administration, a useful device is a small, hard bottle to which a metered dose sprayer is attached. In one embodiment, the metered dose is delivered by drawing the pharmaceutical composition of the present invention solution into a chamber of defined volume, which chamber has an aperture dimensioned to aerosolize and aerosol formulation by forming a spray when a liquid in the chamber is compressed. The chamber is compressed to administer the pharmaceutical composition of the present invention. In a specific embodiment, the chamber is a piston arrangement. Such devices are commercially available.

Alternatively, a plastic squeeze bottle with an aperture or opening dimensioned to aerosolize an aerosol formulation by forming a spray when squeezed is used. The opening is usually found in the top of the bottle, and the top is generally tapered to partially fit in the nasal passages for efficient administration of the aerosol formulation. Preferably, the nasal inhaler will provide a metered amount of the aerosol formulation, for administration of a measured dose of the drug.

The compounds, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active compounds may be in powder form for constitution with a suitable vehicle, e.g., sterile pyrogen-free water, before use.

The compounds may also be formulated in rectal or vaginal compositions such as suppositories or retention enemas, e.g., containing conventional suppository bases such as cocoa butter or other glycerides.

In addition to the formulations described previously, the compounds may also be formulated as a depot preparation. Such long acting formulations may be formulated with suitable polymeric or hydrophobic materials (for example as an emulsion in an acceptable oil) or ion exchange resins, or as sparingly soluble derivatives, for example, as a sparingly soluble salt.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Suitable liquid or solid pharmaceutical preparation forms are, for example, aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of methods for drug delivery, see Langer, 1990, Science 249, 1527-1533, which is incorporated herein by reference.

The LNR-HD domains and optionally other therapeutics may be administered per se (neat) or in the form of a pharmaceutically acceptable salt. When used in medicine the salts should be pharmaceutically acceptable, but non-pharmaceutically acceptable salts may conveniently be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

Suitable buffering agents include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v).

The pharmaceutical compositions of the invention contain an effective amount of a LNR-HD domain and optionally therapeutic agents included in a pharmaceutically-acceptable carrier. The term pharmaceutically-acceptable carrier means one or more compatible solid or liquid filler, diluents or encapsulating substances which are suitable for administration to a human or other vertebrate animal. The term carrier denotes an organic or inorganic ingredient, natural or synthetic, with which the active ingredient is combined to facilitate the application. The components of the pharmaceutical compositions also are capable of being commingled with the compounds of the present invention, and with each other, in a manner such that there is no interaction which would substantially impair the desired pharmaceutical efficiency.

The therapeutic agent(s), including specifically but not limited to the LNR-HD domain, may be provided in particles. Particles as used herein means nano or microparticles (or in some instances larger) which can consist in whole or in part of the LNR-HD domain or the other therapeutic agent(s) as described herein. The particles may contain the therapeutic agent(s) in a core surrounded by a coating, including, but not limited to, an enteric coating. The therapeutic agent(s) also may be dispersed throughout the particles. The therapeutic agent(s) also may be adsorbed into the particles. The particles may be of any order release kinetics, including zero order release, first order release, second order release, delayed release, sustained release, immediate release, and any combination thereof, etc. The particle may include, in addition to the therapeutic agent(s), any of those materials routinely used in the art of pharmacy and medicine, including, but not limited to, erodible, nonerodible, biodegradable, or nonbiodegradable material or combinations thereof. The particles may be microcapsules which contain the LNR-HD domain in a solution or in a semi-solid state. The particles may be of virtually any shape.

Both non-biodegradable and biodegradable polymeric materials can be used in the manufacture of particles for delivering the therapeutic agent(s). Such polymers may be natural or synthetic polymers. The polymer is selected based on the period of time over which release is desired. Bioadhesive polymers of particular interest include bioerodible hydrogels described by Sawhney et. al., 1993, Macromolecules 26, 581-587, the teachings of which are incorporated herein. These include polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

The therapeutic agent(s) may be contained in controlled release systems. The term “controlled release” is intended to refer to any drug-containing formulation in which the manner and profile of drug release from the formulation are controlled. This refers to immediate as well as non-immediate release formulations, with non-immediate release formulations including but not limited to sustained release and delayed release formulations. The term “sustained release” (also referred to as “extended release”) is used in its conventional sense to refer to a drug formulation that provides for gradual release of a drug over an extended period of time, and that preferably, although not necessarily, results in substantially constant blood levels of a drug over an extended time period. The term “delayed release” is used in its conventional sense to refer to a drug formulation in which there is a time delay between administration of the formulation and the release of the drug there from. “Delayed release” may or may not involve gradual release of drug over an extended period of time, and thus may or may not be “sustained release.”

Use of a long-term sustained release implant may be particularly suitable for treatment of chronic conditions. “Long-term” release, as used herein, means that the implant is constructed and arranged to deliver therapeutic levels of the active ingredient for at least 7 days, and preferably 30-60 days. Long-term sustained release implants are well-known to those of ordinary skill in the art and include some of the release systems described above.

Computational techniques can be used to screen, identify, select, and design compounds capable of binding to the Notch1 LNR-HD domain. In particular, computational techniques can be used to identify or design ligands, such as agonists and/or antagonists, that associate with the Notch1 LNR-HD domain. Once identified and screened for biological activity, these agonists, antagonists, and combinations thereof, may be used therapeutically, for example, to decrease Notch1 activity and thus prevent the onset and/or further progression of diseases associated with Notch1 activity. Data stored in a machine-readable storage medium that is capable of displaying a graphical three-dimensional representation of the structure of the potential therapeutic compound or a structurally homologous molecule or molecular complex, as identified herein, or portions thereof may thus be advantageously used for drug discovery. The structure coordinates of the potential therapeutic compounds are used to generate a three-dimensional image that can be computationally fit to the three-dimensional image of the Notch1 LNR-HD domain. The three-dimensional molecular structure encoded by the data in the data storage medium can then be computationally evaluated for its ability to associate with the potential therapeutic compound. When the molecular structures encoded by the data is displayed in a graphical three-dimensional representation on a computer screen, the protein structure can also be visually inspected for potential association with the potential therapeutic compound.

One embodiment of the method of drug design involves evaluating the potential association of a candidate therapeutic compound with the Notch LNR-HD domain. The method of drug design thus includes computationally evaluating the potential of a selected ligand to associate with any of the molecules or molecular complexes set forth above. This method includes the steps of: (a) employing computational means, for example, such as a programmable computer including the appropriate software known in the art or as disclosed herein, to perform a fitting operation between the potential therapeutic compound and the Notch; LNR-HD domain and (b) analyzing the results of the fitting operation to quantify the association between the potential therapeutic compound and the Notch; LNR-HD domain. Several methods can be used to screen potential therapeutic compounds for the ability to associate with the Notch; LNR-HD domain. Selected potential therapeutic compounds may be positioned in a variety of orientations associating with the Notch LNR-HD domain. This may be accomplished using software such as QUANTA (Molecular Simulations, Inc., San Diego, Calif., USA.) and SYBYL (TRIPOS, St. Louis, Mo., USA), followed by energy minimization and molecular dynamics with standard molecular mechanics force fields, such as CHARMM (Molecular Simulations, Inc., San Diego, Calif., USA) and AMBER (P. A. Kollman, University of California at San Francisco, San Francisco, Calif., USA).

Any of the biological or biochemical functions mediated by the LNR-HD domain of Notch1 can be used as an endpoint assay to identify an agent that modulates Notch1 activity (a putative therapeutic compound). The assays may include all of the biochemical or biochemical/biological events described herein, in the references-cited herein, incorporated by reference for these endpoint assay targets, and other functions known to those of ordinary skill in the art or that can be readily identified. Compounds can be identified through cellular assays. Cellular assays involve expressing the LNR-HD domain of Notch1 on the cell surface and testing a variety of compounds for their ability to bind to the expressed peptide. The assay may be performed with labeled compounds, facilitating identification of the compound that binds. In another embodiment a biological readout can be used to identify a putative therapeutic compound. Biological readouts include, but are not limited to, the transport of a part of the Notch receptor to the nucleus or a downstream signaling event (including transcription) initiated by contacting a compound to the LNR-HD domain of Notch1. Biological assays will allow for the identification of both agonists and antagonists or inhibitors. Competition binding assays may also be used to discover compounds that interact with the LNR-HD domain of Notch1 (e.g. binding partners and/or ligands). Thus, a compound is exposed to the LNR-HD domain of Notch1 under conditions that allow the compound to bind or to otherwise interact with the polypeptide. An antibody or fragment thereof against the LNR-HD domain of Notch1 is also added to the mixture. If the test compound interacts with the LNR-HD domain of Notch1, it decreases the amount of antibody that can bind to the LNR-HD domain of Notch1. To perform cell free drug screening assays, it is sometimes desirable to immobilize the LNR-HD domain of Notch1, or its target molecule to facilitate separation of complexes from uncomplexed forms of one or both of the proteins, as well as to accommodate automation of the assay. Agents that modulate the LNR-HD domain of Notch1 of the present invention can be identified using one or more of the above assays, alone or in combination. It is generally preferable to use a cell-based or cell free system first and then confirm activity in an animal or other model system. Such model systems are well known in the art and can readily be employed in this context.

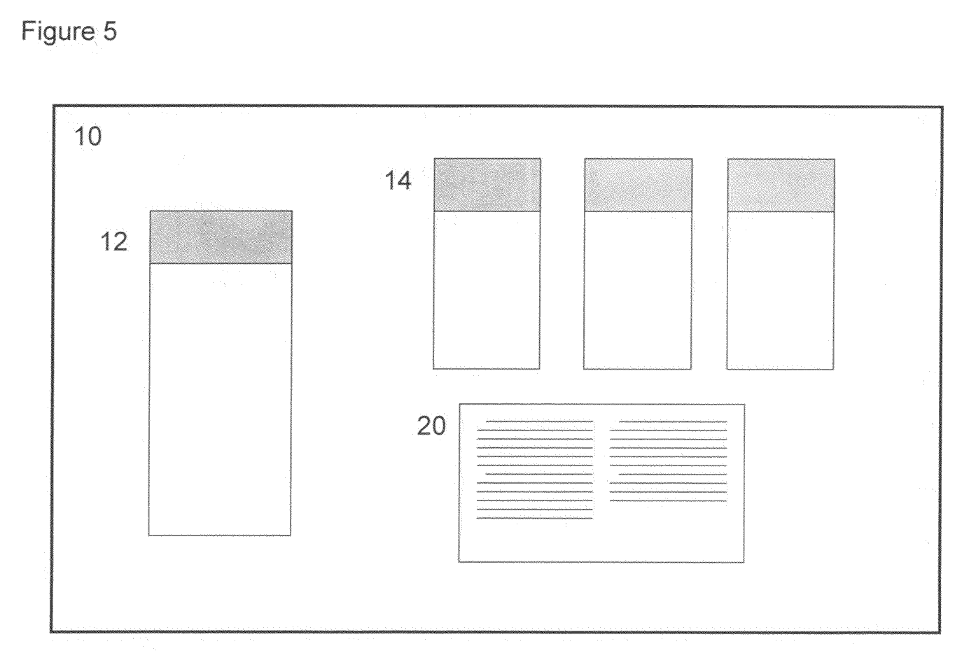

FIG. 5 shows a kit according to the invention. The kit 10 includes a LNR-HD peptide, antibody or nucleic acid 12. The kit 10 may also contain one or more vials or containers 14. The kit also includes instructions 20 for administering the component(s) to a subject who is a disease described herein such as cancer or who has symptoms of such a disease.

In some aspects of the invention, the kit 10 can include a pharmaceutical preparation vial, a pharmaceutical preparation diluent vial, and LNR-HD domain. The vial containing the diluent for the pharmaceutical preparation is optional. The diluent vial contains a diluent such as physiological saline for diluting what could be a concentrated solution or lyophilized powder of LNR-HD domain. The instructions can include instructions for mixing a particular amount of the diluent with a particular amount of the concentrated pharmaceutical preparation, whereby a final formulation for injection or infusion is prepared. The instructions may include instructions for use in a syringe or other administration device. The instructions 20 can include instructions for treating a patient with an effective amount of LNR-HD domain. It also will be understood that the containers containing the preparations, whether the container is a bottle, a vial with a septum, an ampoule with a septum, an infusion bag, and the like, can contain indicia such as conventional markings which change color when the preparation has been autoclaved or otherwise sterilized.

LNR-HD domains can be combined with other therapeutic agents. The LNR-HD domain and other therapeutic agent may be administered simultaneously or sequentially. When the other therapeutic agents are administered simultaneously they can be administered in the same or separate formulations, but are administered at the same time. The other therapeutic agents are administered sequentially with one another and with LNR-HD domain, when the administration of the other therapeutic agents and the LNR-HD domain is temporally separated. The separation in time between the administration of these compounds may be a matter of minutes or it may be longer. Other therapeutic agents include but are not limited to anti cancer therapies.

Thus, the LNR-HD domains may also be administered in conjunction with an anti-cancer therapy. Anti-cancer therapies include cancer medicaments, radiation and surgical procedures. As used herein, a “cancer medicament” refers to an agent which is administered to a subject for the purpose of treating a cancer. As used herein, “treating cancer” includes preventing the development of a cancer, reducing the symptoms of cancer, and/or inhibiting the growth of an established cancer. In other aspects, the cancer medicament is administered to a subject at risk of developing a cancer for the purpose of reducing the risk of developing the cancer. Various types of medicaments for the treatment of cancer are described herein. For the purpose of this specification, cancer medicaments are classified as chemotherapeutic agents, immunotherapeutic agents, cancer vaccines, hormone therapy, and biological response modifiers.

The chemotherapeutic agent may be selected from the group consisting of methotrexate, vincristine, adriamycin, cisplatin, non-sugar containing chloroethylnitrosoureas, 5-fluorouracil, mitomycin C, bleomycin, doxorubicin, dacarbazine, taxol, fragyline, Meglamine GLA, valrubicin, carmustaine and poliferposan, MM1270, BAY 12-9566, RAS farnesyl transferase inhibitor, farnesyl transferase inhibitor, MMP, MTA/LY231514, LY264618/Lometexol, Glamolec, CI-994, TNP-470, Hycamtin/Topotecan, PKC412, Valspodar/PSC833, Novantrone/Mitroxantrone, Metaret/Suramin, Batimastat, E7070, BCH-4556, CS-682, 9-AC, AG3340, AG3433, Incel/VX-710, VX-853, ZDO101, ISI641, ODN 698, TA 2516/Marmistat, BB2516/Marmistat, CDP 845, D2163, PD183805, DX8951f, Lemonal DP 2202, FK 317, Picibanil/OK-432, AD 32/Valrubicin, Metastron/strontium derivative, Temodal/Temozolomide, Evacet/liposomal doxorubicin, Yewtaxan/Paclitaxel, Taxol/Pacitaxel, Xeload/Capecitabine, Furtulon/Doxifluridine, Cyclopax/oral paclitaxel, Oral Taxoid, SPU-077/Cisplatin, HMR 1275/Flavopiridol, CP-358 (774)/EGFR, CP-609 (754)/RAS oncogene inhibitor, BMS-182751/oral platinum, UFT(Tegafur/Uracil), Ergamisol/Levamisole, Eniluracil/776C85/5FU enhancer, Campto/Levamisole, Camptosar/Irinotecan, Tumodex/Ralitrexed, Leustatin/Cladribine, Paxex/Paclitaxel, Doxil/liposomal doxorubicin, Caelyx/liposomal doxorubicin, Fludara/Fludarabine, Pharmarubicin/Epirubicin, DepoCyt, ZD1839, LU 79553/Bis-Naphtalimide, LU 103793/Dolastain, Caetyx/liposomal doxorubicin, Gemzar/Gemcitabine, ZD 0473/Anormed, YM 116, Iodine seeds, CDK4 and CDK2 inhibitors, PARP inhibitors, D4809/Dexifosamide, Ifes/Mesnex/Ifosamide, Vumon/Teniposide, Paraplatin/Carboplatin, Plantinol/cisplatin, Vepeside/Etoposide, ZD 9331, Taxotere/Docetaxel, prodrug of guanine arabinoside, Taxane Analog, nitrosoureas, alkylating agents such as melphelan and cyclophosphamide, Aminoglutethimide, Asparaginase, Busulfan, Carboplatin, Chlorombucil, Cytarabine HCl, Dactinomycin, Daunorubicin HCl, Estramustine phosphate sodium, Etoposide (VP16-213), Floxuridine, Fluorouracil (5-FU), Flutamide, Hydroxyurea (hydroxycarbamide), Ifosfamide, Interferon Alfa-2a, Alfa-2b, Leuprolide acetate (LHRH-releasing factor analogue), Lomustine (CCNU), Mechlorethamine HCl (nitrogen mustard), Mercaptopurine, Mesna, Mitotane (o.p′-DDD), Mitoxantrone HCl, Octreotide, Plicamycin, Procarbazine HCl, Streptozocin, Tamoxifen citrate, Thioguanine, Thiotepa, Vinblastine sulfate, Amsacrine (m-AMSA), Azacitidine, Erthropoietin, Hexamethylmelamine (HMM), Interleukin 2, Mitoguazone (methyl-GAG; methyl glyoxal bis-guanylhydrazone; MGBG), Pentostatin (2′deoxycoformycin), Semustine (methyl-CCNU), Teniposide (VM-26) and Vindesine sulfate, but it is not so limited.

The immunotherapeutic agent may be selected from the group consisting of Ributaxin, Herceptin, Quadramet, Panorex, IDEC-Y2B8, BEC2, C225, Oncolym, SMART M195, ATRAGEN, Ovarex, Bexxar, LDP-03, ior t6, MDX-210, MDX-11, MDX-22, OV103, 3622W94, anti-VEGF, Zenapax, MDX-220, MDX-447, MELIMMUNE-2, MELIMMUNE-1, CEACIDE, Pretarget, NovoMAb-G2, TNT, Gliomab-H, GNI-250, EMD-72000, LymphoCide, CMA 676, Monopharm-C, 4B5, ior egf.r3, ior c5, BABS, anti-FLK-2, MDX-260, ANA Ab, SMART 1D10 Ab, SMART ABL 364 Ab and ImmuRAIT-CEA, but it is not so limited.

The cancer vaccine may be selected from the group consisting of EGF, Anti-idiotypic cancer vaccines, Gp75 antigen, GMK melanoma vaccine, MGV ganglioside conjugate vaccine, Her2/neu, Ovarex, M-Vax, O-Vax, L-Vax, STn-KHL theratope, BLP25 (MUC-1), liposomal idiotypic vaccine, Melacine, peptide antigen vaccines, toxin/antigen vaccines, MVA-based vaccine, PACIS, BCG vacine, TA-HPV, TA-CIN, DISC-virus and ImmuCyst/TheraCys, but it is not so limited.

The present invention is further illustrated by the following Examples, which in no way should be construed as further limiting. The entire contents of all of the references (including literature references, issued patents, published patent applications, and co-pending patent applications) cited throughout this application are hereby expressly incorporated by reference.

### EXAMPLES

TABLE 1
Human Notch1 sequences:

1. Human Notch1 LNR-HD full-length (1447-1734):

Seq ID No.1

MGSSHHHHHHSSGLVPRGSHMAGSENLYFQGEEACELPECQEDAGNKVCS
LQCNNHACGWDGGDCSLNFNDPWKNCTQSLQCWKYFSDGHCDSQCNSAGC
LFDGFDCQRAEGQCNPLYDQYCKDHFSDGHCDQGCNSAECEWDGLDCAEH
VPERLAAGTLVVVVLMPPEQLRNSSFHFLRELSRVLHTNVVFKRDAHGQQ
MIFPYYGREEELRKHPIKRAAEGWAAPDALLGQVKASLLPGGSEGGRRRR
ELDPMDVRGSIVYLEIDNRQCVQASSQCFQSATDVAAFLGALASLGSLNI
PYKIEAVQSETVEPPPPAQ
2. Human Notch1 LNR-HD internal deletion-del31:
(1447-1634, 1666-1734)

Seq ID No.2

MGSSHHHHHHSSGLVPRGSHMAGSENLYFQGEEACELPECQEDAGNKVCS
LQCNNHACGWDGGDCSLNFNDPWKNCTQSLQCWKYFSDGHCDSQCNSAGC
LFDGFDCQRAEGQCNPLYDQYCKDHFSDGHCDQGCNSAECEWDGLDCAEH
VPERLAAGTLVVVVLMPPEQLRNSSFHFLRELSRVLHTNVVFKRDAHGQQ
MIFPYYGREEELRKHPIKRELDPMDVRGSIVYLEIDNRQCVQASSQCFQS
ATDVAAFLGALASLGSLNIPYKIEAVQSETVEPPPPAQ
3. Human Notch1 LNR-HD internal deletion-del29:
(1447-1632, 1662-1734)

Seq ID No.3

MGSSHHHHHHSSGLVPRGSHMAGSENLYFQGEEACELPECQEDAGNKVCS
LQCNNHACGWDGGDCSLNFNDPWKNCTQSLQCWKYFSDGHCDSQCNSAGC
LFDGFDCQRAEGQCNPLYDQYCKDHFSDGHCDQGCNSAECEWDGLDCAEH
VPERLAAGTLVVVVLMPPEQLRNSSFHFLRELSRVLHTNVVFKRDAHGQQ
MIFPYYGREEELRKHPIRRRRELDPMDVRGSIVYLEIDNRQCVQASSQCF
QSATDVAAFLGALASLGSLNIPYKIEAVQSETVEPPPPAQ
4. Human Notch1 LNR-HD internal deletion-del34:
(1447-1627, 1662-1734)

Seq ID No.4

MGSSHHHHHHSSGLVPRGSHMAGSENLYFQGEEACELPECQEDAGNKVCS
LQCNNHACGWDGGDCSLNFNDPWKNCTQSLQCWKYFSDGHCDSQCNSAGC
LFDGFDCQRAEGQCNPLYDQYCKDHFSDGHCDQGCNSAECEWDGLDCAEH
VPERLAAGTLVVVVLMPPEQLRNSSFHFLRELSRVLHTNVVFKRDAHGQQ
MIFPYYGREEELRRRRELDPMDVRGSIVYLEIDNRQCVQASSQCFQSATD
VAAFLGALASLGSLNIPYKIEAVQSETVEPPPPAQ
5. Human Notch1 LNR-HD internal deletion-delpp:
(1447-1630, 1670-1734)

Seq ID No.5

MGSSHHHHHHSSGLVPRGSHMAGSENLYFQGEEACELPECQEDAGNKVCS
LQCNNHACGWDGGDCSLNFNDPWKNCTQSLQCWKYFSDGHCDSQCNSAGC
LFDGFDCQRAEGQCNPLYDQYCKDHFSDGHCDQGCNSAECEWDGLDCAEH
VPERLAAGTLVVVVLMPPEQLRNSSFHFLRELSRVLHTNVVFKRDAHGQQ
MIFPYYGREEELRKHMDVRGSIVYLEIDNRQCVQASSQCFQSATDVAAFL
GALASLGSLNIPYKIEAVQSETVEPPPPAQ
6. Human Notch1 LNR-HD internal deletion-delka:
(1447-1649, 1652-1734)

Seq ID No.6

MGSSHHHHHHSSGLVPRGSHMAGSENLYFQGEEACELPECQEDAGNKVCS
LQCNNHACGWDGGDCSLNFNDPWKNCTQSLQCWKYFSDGHCDSQCNSAGC
LFDGFDCQRAEGQCNPLYDQYCKDHFSDGHCDQGCNSAECEWDGLDCAEH
VPERLAAGTLVVVVLMPPEQLRNSSFHFLRELSRVLHTNVVFKRDAHGQQ
MIFPYYGREEELRKHPIKRAAEGWAAPDALLGQVSLLPGGSEGGRRRREL
DPMDVRGSIVYLEIDNRQCVQASSQCFQSATDVAAFLGALASLGSLNIPY
KIEAVQSETVEPPPPAQ
7. Human Notch1 LNR-HD internal deletion-del11:
(1447-1640, 1652-1734)

Seq ID No.7

MGSSHHHHHHSSGLVPRGSHMAGSENLYFQGEEACELPECQEDAGNKVCS
LQCNNHACGWDGGDCSLNFNDPWKNCTQSLQCWKYFSDGHCDSQCNSAGC
LFDGFDCQRAEGQCNPLYDQYCKDHFSDGHCDQGCNSAECEWDGLDCAEH
VPERLAAGTLVVVVLMPPEQLRNSSFHFLRELSRVLHTNVVFKRDAHGQQ
MIFPYYGREEELRKHPIKRAAEGWASLLPGGSEGGRRRRELDPMDVRGSI
VYLEIDNRQCVQASSQCFQSATDVAAFLGALASLGSLNIPYKIEAVQSET
VEPPPPAQ
8. Human Notch1 LNR-HD internal deletion-de11ee:
(1447-1622, 1634-1734)

Seq ID No.8

MGSSHHHHHHSSGLVPRGSHMAGSENLYFQGEEACELPECQEDAGNKVCS
LQCNNHACGWDGGDCSLNFNDPWKNCTQSLQCWKYFSDGHCDSQCNSAGC
LFDGFDCQRAEGQCNPLYDQYCKDHFSDGHCDQGCNSAECEWDGLDCAEH
VPERLAAGTLVVVVLMPPEQLRNSSFHFLRELSRVLHTNVVFKRDAHGQQ
MIFPYYGRAAEGWAAPDALLGQVKASLLPGGSEGGRRRRELDPMDVRGSI
VYLEIDNRQCVQASSQCFQSATDVAAFLGALASLGSLNIPYKIEAVQSET
VEPPPPAQ

TABLE 2
Human Notch2 Sequences

1. Initial construct: pN2 (with uncleavable C-
terminal His-tag)

Seq ID No. 9

MDQPENLAEGTLVIVVLMPPEQLLQDARSFLRALGTLLHTNLRIKRDSQG
ELMVYPYYGEKSAAMKKQRMTRRSLPGEQEQEVAGSKVFLEIDNRQCVQD
SDHCFKNTDAAAALLASHAIQGTLSYPLVSVVSESLTPERTQLEHHHHHH
Stop
2. Modified initial construct: pN2B (with
cleavable N-terminal His-tag) (1537-1678)

Seq ID No. 10

MHHHHHHGSENLYFQGDQPENLAEGTLVIVVLMPPEQLLQDARSFLRALG
TLLHTNLRIKRDSQGELMVYPYYGEKSAAMKKQRMTRRSLPGEQEQEVAG
SKVFLEIDNRQCVQDSDHCFKNTDAAAALLASHAIQGTLSYPLVSVVSES
LTPERTQLStop
3. N-terminal truncation: pN2B-N1 (1540-1678)

Seq ID No. 11

MHHHHHHGSENLYFQGENLAEGTLVIVVLMPPEQLLQDARSFLRALGTLL
HTNLRIKRDSQGELMVYPYYGEKSAAMKKQRMTRRSLPGEQEQEVAGSKV
FLEIDNRQCVQDSDHCFKNTDAAAALLASHAIQGTLSYPLVSVVSESLTP
ERTQLStop
4. Internal Deletion: pN2B del: (1540-1595 and
1618-1678)

Seq ID No. 12

MHHHHHHGSENLYFQGENLAEGTLVIVVLMPPEQLLQDARSFLRALGTLL
HTNLRIKRDSQGELMVYPYYGEVAGSKVFLEIDNRQCVQDSDHCFKNTDA
AAALLASHAIQGTLSYPLVSVVSESLTPERTQLStop
5. Internal Deletion with RRRR reintroduced: pN2B-
RRRR: (1540-1595 and 1618-1678)

Seq ID No. 13

MHHHHHHGSENLYFQGENLAEGTLVIVVLMPPEQLLQDARSFLRALGTLL
HTNLRIKRDSQGELMVYPYYGRRRREVAGSKVFLEIDNRQCVQDSDHCFK
NTDAAAALLASHAIQGTLSYPLVSVVSESLTPERTQLStop
6. Internal Deletion with RRRRG reintroduced:
pN2B-RRRRG: (1540-1595 and 1618-1678)

Seq ID No. 14

MHHHHHHGSENLYFQGENLAEGTLVIVVLMPPEQLLQDARSFLRALGTLL
HTNLRIKRDSQGELMVYPYYGRRRRGEVAGSKVFLEIDNRQCVQDSDHCF
KNTDAAAALLASHAIQGTLSYPLVSVVSESLTPERTQLStop
7. hNotch2 LNR-HD: (pN2FL) (1423-1677):

Seq ID No. 15

MGSSHHHHHHSSGLVPRGSHMENLYFQGATCLSQYCADKARDGVCDEACN
SHACQWDGGDCSLTMENPWANCSSPLPCWDYINNQCDELCNTVECLFDNF
ECQGNSKTCKYDKYCADHFKDNHCNQGCNSEECGWDGLDCAADQPENLAE
GTLVIVVLMPPEQLLQDARSFLRALGTLLHTNLRIKRDSQGELMVYPYYG
EKSAAMKKQRMTRRSLPGEQEQEVAGSKVFLEIDNRQCVQDSDHCFKNTD
AAAALLASHAIQGTLSYPLVSVVSESLTPERTQStop
8. hNotch2 LNR-HD with internal deletion: (pN2FL-
del) (1423-1595 1618-1677)

Seq ID No. 16

MGSSHHHHHHSSGLVPRGSHMENLYFQGATCLSQYCADKARDGVCDEACN
SHACQWDGGDCSLTMENPWANCSSPLPCWDYINNQCDELCNTVECLFDNF
ECQGNSKTCKYDKYCADHFKDNHCNQGCNSEECGWDGLDCAADQPENLAE
GTLVIVVLMPPEQLLQDARSFLRALGTLLHTNLRIKRDSQGELMVYPYYG
EVAGSKVFLEIDNRQCVQDSDHCFKNTDAAAALLASHAIQGTLSYPLVSV
VSESLTPERTQLStop

TABLE 3
Primers used to generate Human Notch1 constructs

1. Primers to clone LNR-HD from Notch 1, introduce 5′ Nde1,
TEV and 3′ BamH1

| | | | |
|---|---|---|---|
| Seq ID No. 17 | ggaattccatatggcaggctccgagaacctg | lnrhd_tevnde | forw |
| Seq ID No. 18 | cgggggcggccgcgtcattcctaggtggtgg | lnrhd_bam | rev |

2. Quick change primers for deletion constructs:

| | | | |
|---|---|---|---|
| Seq ID No. 19 | gagctgcgcaagcacatggacgtccg | delpp | forw |
| Seq ID No. 20 | ctcgacgcgttcgtgtacctgcaggc | delpp | rev |
| Seq ID No. 21 | ctgggccaggtgtcgctgctccctg | delka | forw |
| Seq ID No. 22 | gacccggtccacagcgacgagggac | delka | rev |
| Seq ID No. 23 | ctacggccgcgccgccgagg | del11ee | forw |
| Seq ID No. 24 | gatgccggcgcggcggctcc | del11ee | rev |
| Seq ID No. 25 | gggctgggcctcgctgctccc | del11_2 | forw |
| Seq ID No. 26 | cccgacccggagcgacgaggg | del11_2 | rev |
| Seq ID No. 27 | gaggagctgcgccggcggagggag | del34_2 | forw |
| Seq ID No. 28 | ctcctcgacgcggccgcctccctc | del34_2 | rev |

TABLE 4
Primers used to generate Human Notch 2 constructs

1. Primers to clone LNR-HD from Notch2, introduce 5′ Nde1, TEV and 3′BamH1

| | | | |
|---|---|---|---|
| Seq ID No. 29 | aattccatatggagaacctgtattttcagggcgccacctgtctgagccag | hN2 | forw |
| Seq ID No. 30 | actgaggtcttgcgtgagtcattcctaggtggtgg | hN2 | rev |

2. Quick change primers for deletion constructs:

| | | | |
|---|---|---|---|
| Seq ID No. 31 | gtacccctattatggtgaggtggctggctctaaag | 22aadel | forw |
| Seq ID No. 32 | catggggataataccactccaccgaccgagatttc | 22aadel | rev |
| Seq ID No. 33 | gtacccctattatggtcgccgtcgccgtgaggtggctggctct | RRRR | forw |
| Seq ID No. 34 | catggggataatccagcggcagcggcactccaccgaccgaga | RRRR | rev |
| Seq ID No. 35 | gtacccctattatggtcgccgtcgccgtggcgaggtggctggctc | RRRRG | forw |
| Seq ID No. 36 | catggggataataccagcggcagcggcaccgctccaccgaccgag | RRRRG | rev |
| Seq ID No. 37 | ggaattccatatggaccaacctgagaacctggc | pN2 | forw |
| Seq ID No. 38 | actgaggtcttgcgtgagtcggagctcgcc | pN2 | rev |
| Seq ID No. 39 | acggatccgagaacctgtactttcaaggtgaccaacctgagaacctggc | pN2B | forw |
| Seq ID No. 40 | gaggtcttgcgtgagtcgagattgagctcgcc | pN2B | rev |
| Seq ID No. 41 | ggaattccatatgcaccaccaccaccaccacggatccgagaacctgt | H6_Ta_pN2 | f |
| Seq ID No. 42 | tcgagattgagctcgcc | H6_Ta_pN2 | r |
| Seq ID No. 43 | cctgtactttcaaggtgagaacctggcagaag | QC_pN2_N1 | f |
| Seq ID No. 44 | ggacatgaaagttccactcttggaccgtcttc | QC_pN2_N1 | r |

### Example 1

### Cloning, Expression, and Purification of Human Notch1 and 2 Full-Length Negative Regulatory Region Constructs

### Cloning:

The wildtype full-length Negative Regulatory Region constructs for human Notch1 (E1447-Q1734; Seq ID No. 1) and human Notch2 (A1423-Q1677; Seq ID No. 15) were subcloned into the plasmid vector pET-15b(+) (Novagen, San Diego, USA) with an N terminal TEV protease recognition site (GSENLYFQG; Seq ID No. 45) engineered between the plasmid derived His-tag for affinity purification and the Notch sequence of interest.

Construction of the Human Notch1 and 2 Constructs with Internal Deletions:

The corresponding wild type construct was used as a template to obtain the hNotch1 and hNotch2 deletion constructs (Table 1 and Table 2). Internal deletions were introduced with quick-change primers (Table 3 and Table 4) (QuickChange Site-Directed Mutagenesis Kit (Stratagene, San Diego, USA)).

### Expression and Purification of the Full-Length Negative Regulatory Region Human Notch1 and 2 Constructs:

Expression plasmids were transformed into Rosetta(DE3)pLysS Escherichia coli strain cells (Novagen, San Diego, USA). A single colony carrying the plasmid containing the protein of interest was used to inoculate Luria-Bertani Broth. The cell cultures grown at 37° C. were induced for protein expression by addition of 0.4 mM isopropyl-1-thio-β-D-galactopyranoside at an OD₆₀₀=0.7. After induction for 4 h, cells were harvested by centrifugation at 4° C., 5000 g. Each 2L cell pellet was resuspended in 25 ml of Lysis Buffer 1 (50 mM Tris pH 8.0, 300 mM NaCl, 20% sucrose, 10 mM BME) and frozen at −20° C. overnight. Cells were lysed by thawing, followed by three cycles of sonication for 30 s on ice after which the insoluble protein (including the protein of interest) was pelleted by ultracentrifugation at 40,000 g for 30 min. The pellet was resuspended in 25 ml Lysis Buffer 2 (50 mM Tris pH 8.0, 6M Urea) and the supernatant containing the protein was collected after 30 min. ultracentrifugation at 40,000 g. The supernatant was incubated with nickel-nitrilotriacetic acid (Ni-NTA) agarose resin (Qiagen, Valencia, USA) at room temperature for 2 h. Ni-NTA agarose resin containing the bound protein was pelleted at 500 g for 5 min and resuspended in 50 ml of Wash Buffer 1 (50 mM Tris pH 8.0, 300 mM NaCl, 2.5 M Urea, 10 mM immidazole) to remove nonspecifically bound proteins, and poured into an empty column. The beads were washed once more with 50 ml of Wash Buffer 2 (50 mM Tris pH 8.0, 300 mM NaCl, 1 M Urea) and the bound His-tagged Notch constructs were eluted from the Ni-NTA agarose resin with Elution Buffer (50 mM Tris pH 8.0, 300 mM NaCl, 1 M Urea, 500 mM immidazole). The eluted protein was incubated with in house produced N-terminally His-tagged TEV protease in the presence of 2 mM DTT at room temperature for 12-16 h. The completeness of cleavage was verified with SDS PAGE gel and/or HPLC and the cleaved protein was dialyzed against refolding buffer (50 mM Tris, pH 8.0, 250 mM NaCl, 50 mM CaCl₂, 1 mM cystine, 5 mM cysteine) with two buffer changes in 24-48 hrs. After the completion of the folding (as monitored by HPLC) the protein was dialyzed into anion exchange buffer (25 mM Tris pH 8.0, 25 mM NaCl, 5 mM CaCl₂) and further purified on a MONOQ column. The purity and the identity of the purified proteins were verified by SDS PAGE and/or HPLC as well as MALDI-TOF Spectroscopy.

### Example 2

### Cloning, Expression, and Purification of Human Notch2 Heterodimerization Domain (HD) Constructs

### Cloning:

The wildtype hNotch2 construct (D1537-L1678; Seq ID No. 9) was subcloned into the plasmid vector pET-21a(+) (Novagen, San Diego, USA) initially with a C-terminal hexahistidine tag for affinity purification. An N-terminal hexahistidine tag followed by a TEV recognition sequence (GSENLYFQG; Seq ID No. 45) was engineered into the same vector using sequential PCR.

### Construction of Notch2 HD Constructs:

The cleavable N-terminally His-tagged wild type construct (which includes Seq ID No. 10) was used as a template to obtain all the different truncated Notch heterodimerization (HD) constructs (Table 2) using the QuickChange Site-Directed Mutagenesis Kit (Stratagene, San Diego, USA).

### Expression and Purification of the Notch2 HD Constructs:

Notch2 HD expression plasmids were transformed into BL21(DE3)pLysS Escherichia coli strain cells (Novagen, San Diego, USA). A single colony carrying the plasmid containing the protein of interest was used to inoculate Luria-Bertani Broth. The cell cultures grown at 37° C. were induced for protein expression by addition of 0.4 mM isopropyl-1-thio-β-D-galactopyranoside at an OD₆₀₀=0.7. After induction for 4 h, cells were harvested by centrifugation at 4° C., 5000 g. Each 2L cell pellet was resuspended in 25 ml of Lysis Buffer (50 mM Tris pH 8.0, 300 mM NaCl, 20% sucrose, 10 mM BME, and protease inhibitors) and frozen at −20° C. overnight. Cells were lysed by thawing, followed by three cycles of sonication for 30 s on ice and the supernatant containing the soluble protein was collected after ultracentrifugation at 40,000 g. The pellet was resuspended once more in 25 ml of lysis buffer and the procedure was repeated to maximize the protein yield. The supernatants from multiple rounds of ultracentrifugation were pooled together and incubated with nickel-nitrilotriacetic acid (Ni-NTA) agarose resin (Qiagen) at 4° C. for 2 h. Ni-NTA agarose resin containing the bound protein was pelleted at 500 g for 5 min and then resuspended in 50 ml of Wash Buffer (50 mM Tris pH 8.0, 300 mM NaCl, 10 mM immidazole) twice to remove nonspecifically bound proteins. After the second wash, the resin was resuspended in wash buffer once more and poured into an empty column. The bound His-tagged pN2 construct was eluted from the Ni-NTA agarose resin with Elution Buffer (50 mM Tris pH 8.0, 300 mM NaCl, 250 mM immidazole). The eluted protein was incubated with in house produced N-terminally His-tagged TEV protease in the presence of 2 mM DTT at room temperature for 12-16 h. The completeness of cleavage was verified with SDS PAGE gel and/or HPLC and the cleaved protein was dialyzed against FPLC Buffer (50 mM Tris, pH 8.0, 300 mM NaCl) for 12 hrs to allow the formation of the native disulfide bond within the heterodimerization domain. The dialyzed sample was further purified by size exclusion chromatography on a Superdex 75 column (Amersham Biosciences, Piscataway, USA) in the FPLC Buffer. The identity of the purified protein was verified by MALDI.

### Example 3

### Crystallization of Human Notch2 LNR-HD with Internal Deletion (pN2FL-del; Seq ID No 16)

After the initial round of successful crystallization of 10 mg/ml pN2FL-del (Seq ID No. 16) at 100 mM buffer (Bis-Tris pH 6.0-7.0, Hepes pH 7.0-8.0, or Tris pH 8.0-9.0), 200 mM MgCl₂, 14-30% PEG3350, crystallization buffer conditions were refined to 100 mM Tris-HCl pH 8.5, 200 mM MgCl₂, 24% PEG3350, 10% glycerol for the Se-Met labeled protein crystals and 100 mM Bis-Tris pH 6.5, 200 mM MgCl₂, 18% PEG3350, 10% glycerol for native crystals. The cryo-protectant solutions used for data collection at the synchrotron were 100 mM Tris-HCl pH 8.5, 200 mM MgCl₂, 10 mM CaCl₂, 26% PEG3350, 20% glycerol for the Se-Met labeled protein crystals and 100 mM Bis-Tris pH 6.5, 200 mM MgCl₂, 10 mM CaCl₂, 24% PEG3350, 20% glycerol for native crystals. The crystals were grown using 10 mg/ml protein stock (in 25 mM Tris pH 8.0, 100 mM NaCl, 10 mM CaCl₂) either in 1 μl (protein stock)×1 μl (crystallization buffer) or 1.5 μl (protein stock)×2.5 μl (crystallization buffer) as hanging drops. The protein stock was prepared by dialyzing the protein after purification (as described earlier) against 25 mM Tris pH 8.0, 100 mM NaCl, 10 mM CaCl₂ followed by concentration in Vivaspin MWCO 10K Concentrators (Vivascience ISC, Edgewood, N.Y.) to achieve the desired stock concentration as determined by UV absorbance at 280 nM (extinction coefficient 34200). Under the described conditions the protein crystallized into single 3-dimentional single crystals with the longest dimension ranging between 0.1-0.2 μm in 2-5 days.

Datasets to determine the X-ray structure were collected on the X29B beamline at the NSLS (Brookhaven, N.Y.) using crystal to detector distance of 275 mm. All data were collected for 180-360 consecutive frames with 10 oscillation per frame with 3-4s exposure times at each wavelength. The data sets were indexed, integrated, and merged using HKL2000 (HKL Research, Charlottesville, Va.).

Before the data collection of Se-Met crystals, a fluorescence scan of the crystal was performed to confirm the presence of Se in the crystal and to determine the exact location of the absorption edge of the Se. Based on this scan either only the peak wavelength (for SAD data collection) or the following three wavelengths (inflection, peak, and remote) for MAD data collection which would maximize the anomalous and dispersive differences, were chosen.

| | |
|---|---|
| λ₁: | 0.9794 Å (inflection point/absorption edge) |
| λ₂: | 0.9792 Å (peak of the scan) |
| λ₃: | 0.9715 Å (remote wavelength) |

The Se-Met heavy atom sites were independently determined using either two separate MAD data sets or four SAD datasets combined with the best native data set, using the program SHARP (Global Phasing, Cambridge, UK) and an initial partial model containing two molecules per asymmetric unit was built by the Arp-WARP module of SHARP (EMBL, Heidelberg, Germany). This partial model was used to determine the non-crystallographic symmetry which was then used to improve electron density maps.

The NOTCH2 LNR-HD with internal deletion contained two copies in the asymmetric unit. The noncrystallographic symmetry (NCS) between these two molecules was used to calculate NCS averaged maps (Rave, Uppsalla group) to aid in manual building. NCS restraints were relaxed during iterative manual building in Coot (Emsley et al. 2004, Acta Cryst. D. Biol. Cryst. 60, 2126) and refinement with CNS (Brunger et al. 1998, Acta Cryst D. 54, 905-921) and Refmac (Murshudov et al. 1997, Acta Crystal D. 53, 240-245). The final model contains 164 molecules of solvent and 4 of glycerol with an overall R_{cryst}/R_{free} of 22.4/26.8% at 2.0 Å resolution. The percentage of protein residues in core, allowed, generous, and disallowed regions of the Ramachandran plot are 81.3, 16.3, 2.5, and 0.0, respectively.

The data collection, phasing and refeninent statistic are presented in Table 5.

TABLE 5
Data collection, phasing and refinement statistics

| | | Human NOTCH2 |
|---|---|---|
| | | LNR-HD-deletion |
| Data collection | | |
| Space group | | P2₁2₁2₁ |
| Cell dimensions | | |
| a, b, c (Å) | | 45.4, 74.7, 139.4 |
| α, β, γ (°) | | 90, 90, 90 |
| Resolution (Å) | | 30.0-2.0(2.07-2.0)* |
| R_{sym} or R_{merge} | | 7.5(40.5) |
| I/σI | | 17.2(3.2) |
| Completeness (%) | | 97.2(98.1) |
| Redundancy | | 4.9(4.7) |
| Refinement | | |
| Resolution (Å) | | 30.0-2.0 |
| No. reflections | | 31791 |
| R_{work}/R_{free} | | 22.4/26.8 |
| No. atoms | | |
| Protein | | 3411 |
| Ligand/ion | | 7 |
| Water, glycerol | | 164, 60 |
| B-factors | | |
| Protein | (molecule 1 in a.s.u.) | 43 |
| | (molecule 2 in a.s.u.) | 54 |

| | |
|---|---|
| Ligand/ion | 57 |
| Water | 58 |
| R.m.s deviations | |
| Bond lengths (Å) | 0.01 |
| Bond angles (°) | 1.174 |

### Experiment 4: Effect of Interdomain Interactions on Notch Function

### Constructs Used in Cell-Based Reporter Assays:

All Notch expression plasmids were engineered to contain the start codon and the signal peptide of NOTCH1 in the mammalian expression vector pcDNA3.1. NOTCH2/NOTCH1 chimeras were made by ligation of PCR products amplified from a NOTCH2 cDNA into the NOTCH1 ΔEGF cDNA after digestion with the restriction enzymes BamHI and Bsu36I. The resulting chimeric cDNAs encoded polypeptides comprised of the NOTCH1 leader peptide, portions of the ectodomain of NOTCH2, the transmembrane domain of NOTCH2, and the first 13 amino acids of the intracellular domain of NOTCH2 fused to all but the first 13 amino acids of the intracellular domain of NOTCH1. The ΔEGF NOTCH2/NOTCH1 chimera starts with residue Pro 1422 of NOTCH2. Further deletions start with NOTCH2 residue Ser1456 (ΔEGFΔLNR-A), Pro1462 (AEGFALNR-AAlink), Gln1497 (ΔEGFΔLNR-AB), and Ala1535 (ΔEGFΔLNR-ABC). The ΔEGF form of NOTCH1 begins with residue Glu1446 of NOTCH1 after the Bam HI site that follows the signal peptide (Sanchez-Irizarry et al. 2004, Mol Cell Biol 24; 9265-73). Further deletions start with residue Ser1481 (ΔEGFΔLNR-A), Lys1489 (ΔEGFΔLNR-AΔlink), Gln1523 (ΔEGFΔLNR-AB), and His1564 (ΔEGFΔLNR-ABC).

### Cell Based Reporter Assays:

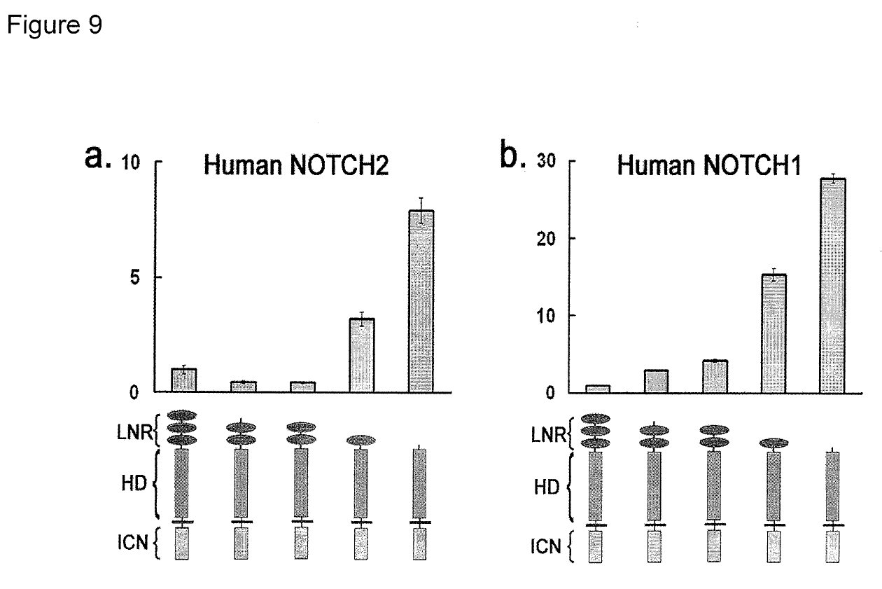

To address the functional importance of the autoinhibitory interdomain interactions, we used the structure to guide the design of nested NOTCH1 and NOTCH2 NRR deletions, and determined the effects of the deletions on receptor activation in cells (FIG. 9a, b). These experiments show that LNR-A, the LNR-A-B linker, and LNR-B must all be removed before activation occurs, consistent with the structural prediction that key interactions of LNR-B and the LNR-AB linker with the HD domain protect the metalloprotease site.

### Overview of Structure

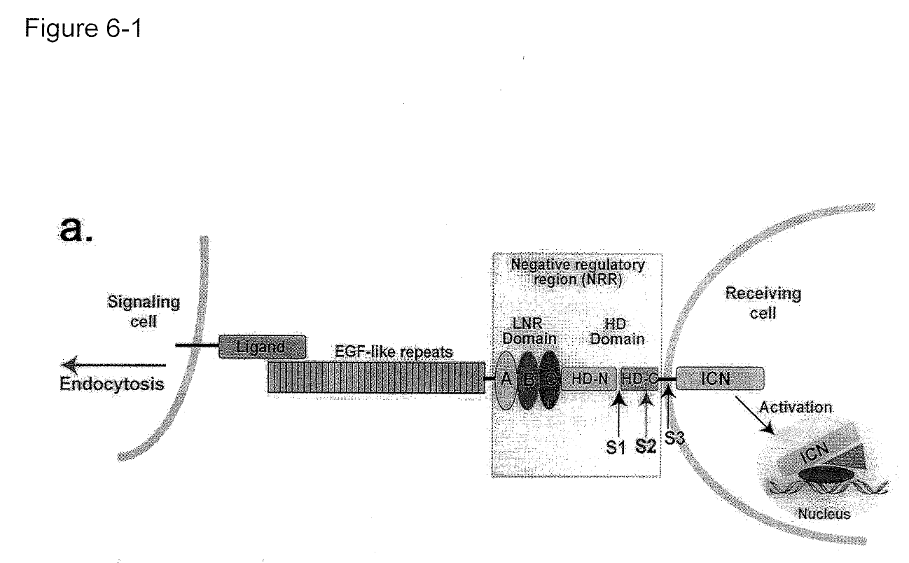

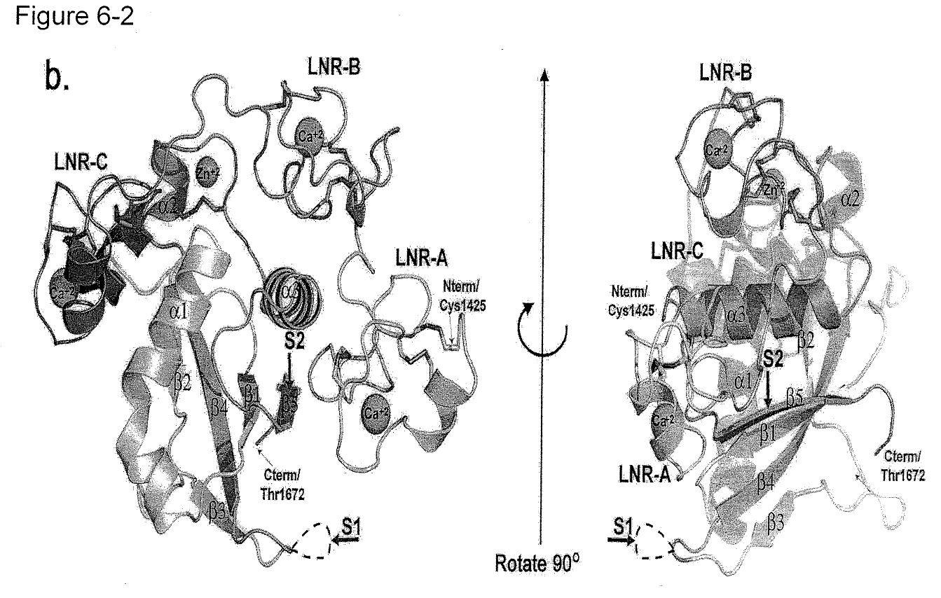

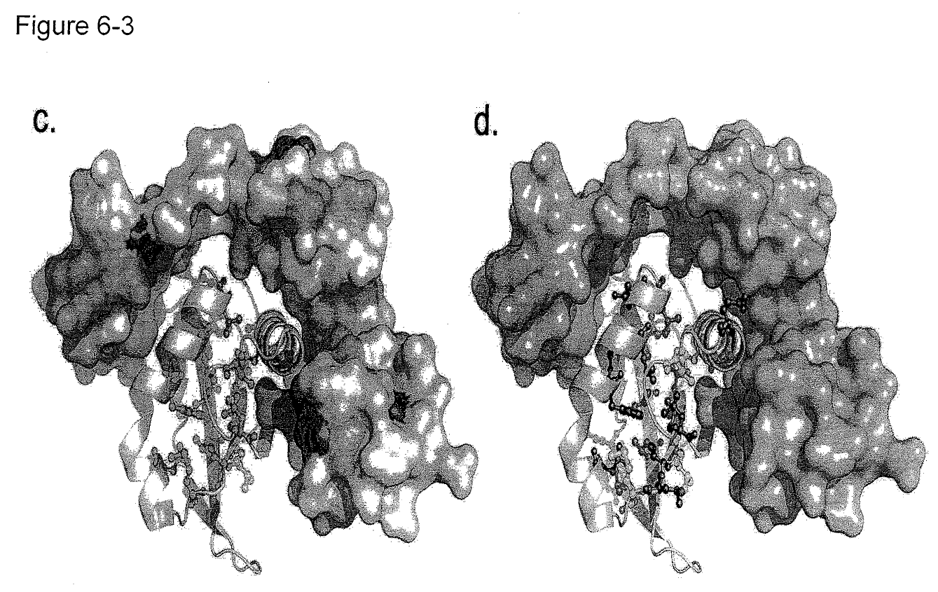

The NRR (LNR-HD) adopts a compact conformation with overall dimensions of 60×45×25 Å (FIG. 6b). Numerous interdomain contacts wrap the three LNR modules around the HD domain, bringing the N- and C-termini of the NRR close together. This arrangement produces a cauliflower-like shape, in which the three LNR module ‘florets’ cover and protect the HD domain ‘stem’ (FIG. 6b).

Like the prototype LNR from NOTCH1 (Vardar et al. 2003, Biochem. 42, 7061), the LNR modules share an irregular fold with little secondary structure. Each contains three disulfide bonds with a characteristic connectivity and a bound Ca⁺⁺ ion coordinated by acidic and polar residues. The first two LNRs (A and B) are connected by a highly conserved, well-ordered ten-residue linker and share a hydrophobic interface formed by the stacking of aromatic residues. In contrast, LNR-C does not make direct contact with either A or B, and the 6-residue linker connecting LNR-B to C is more poorly ordered and not well conserved.

The two halves of the HD domain, normally divided by furin at site S1, form a single protein domain that consists of an intimately intertwined α-β sandwich containing five β-strands (β1-β5) and two α-helices (α1 & α3) connected by several conserved loops and a short helix (α2; FIG. 6b). The secondary structural elements of the HD fold around a conserved hydrophobic core with side chains that project from residues along the concave surface formed by the β-sheet and from the inner faces of the two flanking helices α1 and α3 (FIG. 6c). Remarkably, almost all of the known leukemia-associated mutations in human NOTCH1 map onto this hydrophobic core, underscoring its importance in maintaining the structural integrity of the HD (FIG. 6c, d).

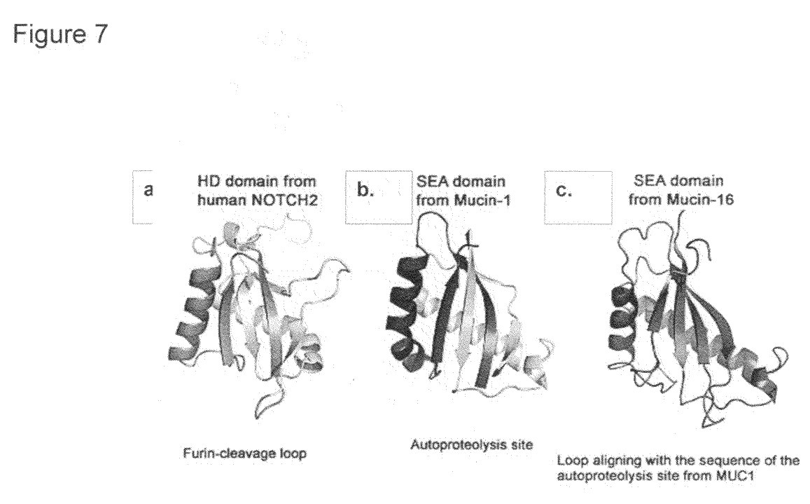

Site S1, which was excised from the crystallized construct, would lie in a loop connecting β-strands 3 and 4, more than 17 Å away from the S2 site and well removed from all other interdomain contacts (FIG. 6). Site S2 lies near the C-terminal end of the HD domain in the middle of the final β-strand (β5) and in close proximity to α3. A structural similarity search using the Dali server (Holm et al. 1996, Science 273, 595) revealed no significant matches for the LNRs and a single hit for the HD domain with Z-score >6: a SEA (sea urchin sperm protein, enterokinase, and grin) domain (pdb code livz) from murine mucin-16 (Maede et al. 2004, J. Biol. Chem. 279, 13174). Of interest, this and homologous mucin SEA domains undergo self-cleavage in the loop connecting strands two and three of the four-stranded sheet (Macao et al. 2006, Nat. Str. Mol Biol 13, 71-76), a position that corresponds structurally to the S1 site of the Notch 2 HD domain (FIG. 7).

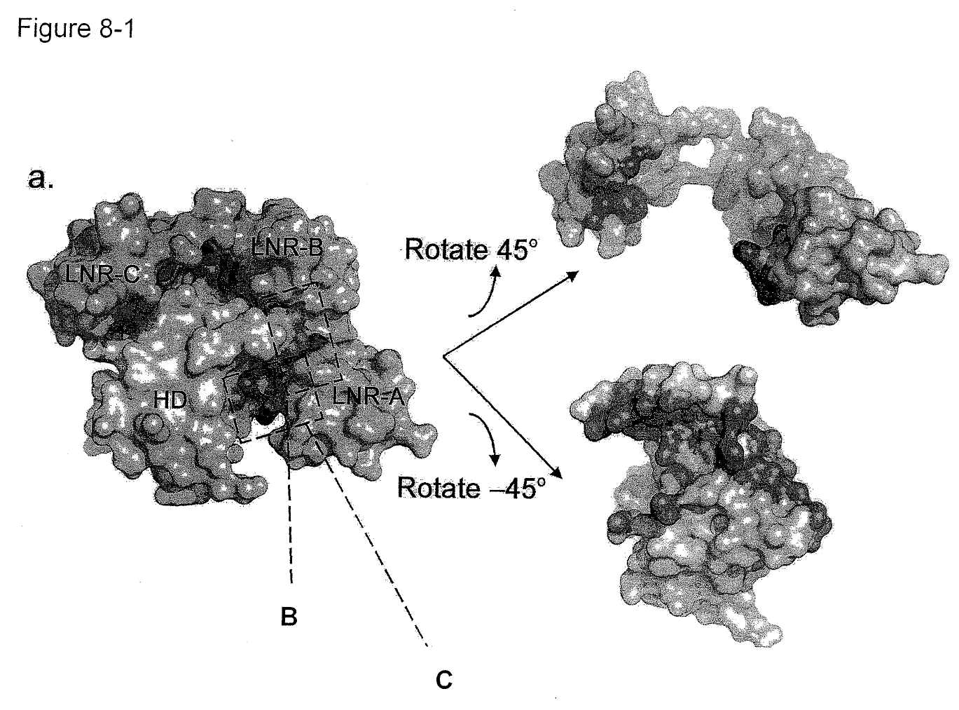

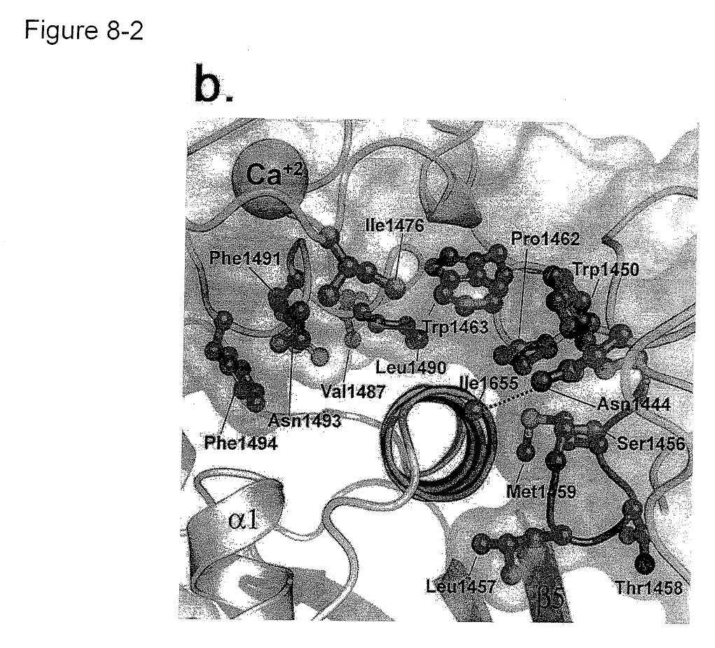

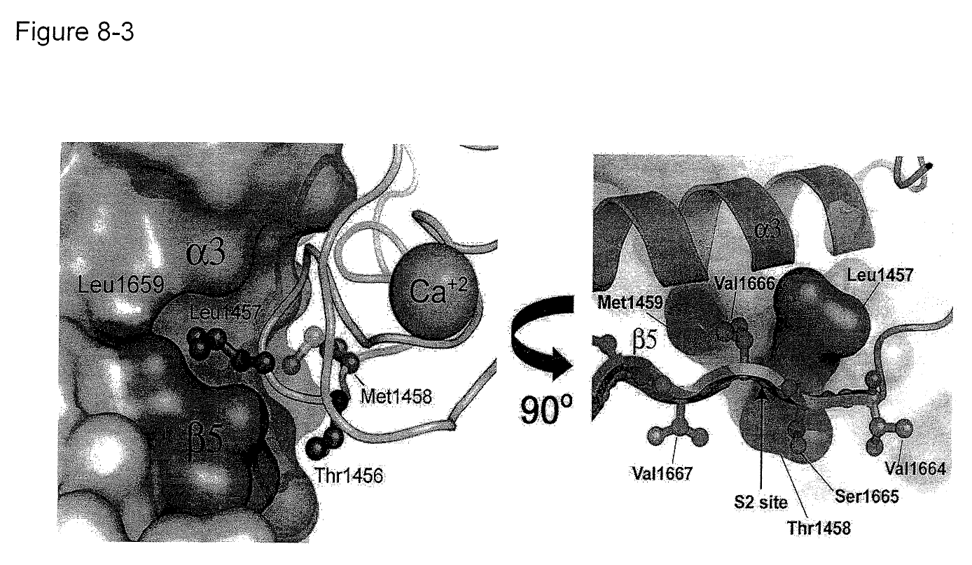

Extensive interactions between the LNR and the HD domains stabilize the NRR in the autoinhibited conformation. The LNRs wrap around the HD domain, masking the S2 site and key structural elements by burying a total solvent accessible surface area of ˜3000 Å² (FIG. 8a), almost half of which derives from interactions between LNR-C and the HD domain. Hydrophobic residues from LNR-C pack against helices 1 and 2 of the HD domain core, whereas electrostatic interactions between LNR-C and the HD domain fix their relative positions. The charged and polar interactions include a conserved salt bridge between Arg1567 and Asp1506, an extensive hydrogen-bonding network, and a Zn²⁺ ion that is coordinated via the side chains of His1574 and His1638 from the HD domain, Glu1525 from LNR-C, and a water molecule. Though it is tempting to propose a role for this ion in the regulation of Zn⁺⁺-dependent ADAM-metalloproteases, the Zn²⁺-binding site is not conserved and may simply stabilize the LNR-C/HD domain interface of NOTCH2. A cluster of conserved residues on the exposed surface of LNR-C is a good candidate region for other intra- or intermolecular interactions (FIG. 6c).

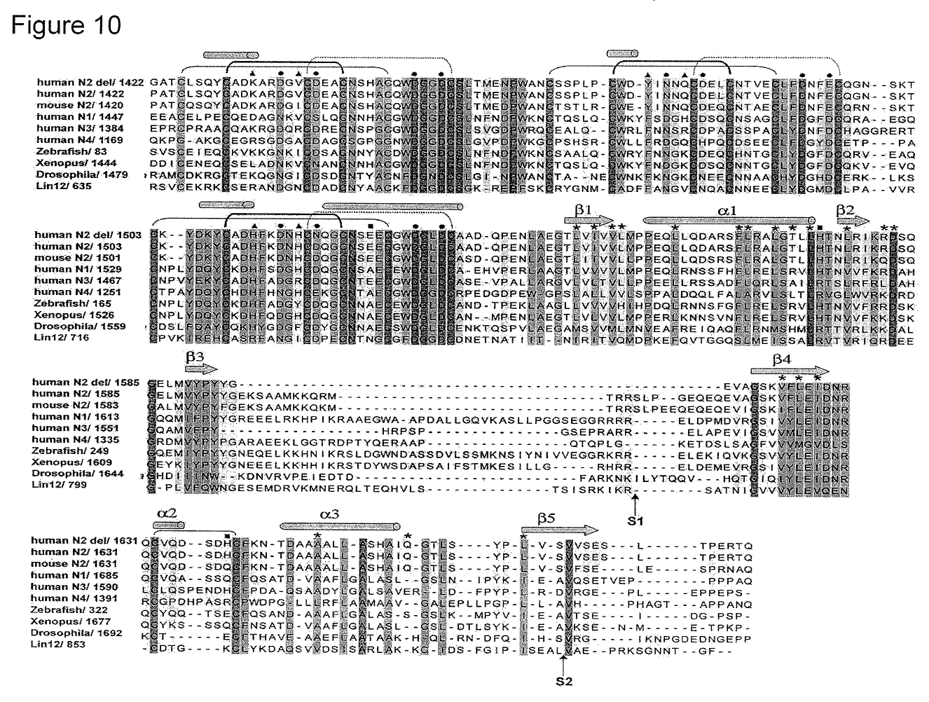

The remaining long-range interdomain contacts result from the packing of LNR-B, the linker connecting LNR-A and LNR-B, and the C-terminal end of LNR-A against the HD domain, which together account for 25, 23, and 5% of the overall LNR-HD interface, respectively. Together, these interactions mask the S2 site and buttress the HD domain α3 helix (FIG. 8b), which is packed against the β-strand housing the S2 site (FIG. 8c). The high degree of sequence conservation among the residues engaged in these key intra- and inter-domain interactions argues that the observed autoinhibited conformation is a general feature of Notch receptors (FIGS. 8 and 10).

The S2 site (FIG. 8c) is nestled at the mouth of a small hydrophobic pocket bounded by β5 (which contains the Ser1665/Val1666 cleavage site), the inner face of β 3, and a highly conserved leucine residue (Leu1659) in the linker that connects these elements. The “plug” that fills the pocket and blocks access to the scissile bond is the side chain of Leu1457, which lies in the linker connecting LNR-A and B (FIG. 8c). Leu1457 is fixed within the pocket by a hydrogen bond between its carbonyl oxygen and the backbone amide of Val 1666 and by packing against residues that line the pocket (Leu1650, Ala1651, Ala1654, Leu1663, and Val1666), which account for 10% of the total LNR/HD domain interface. Together, the side chains of Leu1457, Thr1458 and Met1459 straddle the S2 site and prevent access from either side (FIG. 8c). Precise anchoring of HD domain helix 3 above the S2 site also maintains the pocket. This helix is clamped into position by hydrophobic residues derived from the LNR-AB linker (Met1459, Pro1462 and Trp1463) and LNR-B (Ile1476, Val1487, Leu1490, Asn1493 and Phe1494, see FIG. 8b). In addition, a hydrogen bond between the Asn1444 side chain of LNR-A and the backbone carbonyl group of Ile1655 anchors helix 3 at its C-terminal end. The burial of the S2 site establishes the need for a substantial conformational movement to enable metalloprotease cleavage in response to ligand binding.

### Relationship Between Notch Structure and Function

Our structure provides the first direct experimental evidence that a substantial conformational movement in the NRR is required to expose the S2 site upon ligand binding, and argues strongly against models that require changes in oligomerization or other intermolecular interactions to uncover the S2 site. In addition, the subunits comprising the HD domain are woven into an intimately intertwined α-β sandwich by an extensive H-bonding network, which disfavors models in which ligand binding causes the dissociation of the subunits to expose S2 without first disengaging the LNR domain from the HD domain.

The structure of the autoinhibited conformation does not itself distinguish whether ligand binding causes a simple allosteric conformational change in the NRR, or whether endocytosis of ligand bound to Notch is required to exert a mechanical force to activate the receptor, as first proposed by Parks and Muskavitch (Parks et al. 2000, Development 127, 1373-1385). In the mechanical force model for activation, which we refer to as the “lift and cut” model, force would induce the conformational movement that renders Notch susceptible to S2 cleavage by peeling the protective LNR modules away from the HD domain. Since the ligand binding domain is immediately N-terminal to LNR-A of the NRR, the mechanical tug would first “lift” LNR-A, then LNR-B, and finally LNR-C away from the HD domain. Effective transmission of force to the NRR would require a tight interaction between ligand and receptor, which is supported by direct measurements of adhesion strength in pairs of cells expressing Notch and ligand, respectively (Ahimou et al. 2004, J Cell Biol 167: 1217-1229), and by prior observations of Notch ectdodomain trans-endocytosis into ligand-bearing cells (Parks et al. 2000, Development 127, 1373-1385). The extent of the proposed movement is consistent with prior studies showing that the active site of TNFα-converting enzyme (TACE), a metalloprotease implicated in S2 cleavage of Notch, is buried in a hydrophobic groove that cups the substrate from both sides of the scissile bond (Maskos et al. 1998, PNAS 95: 3408-3412). As a result, it appears that both the hydrophobic plug that fills the pocket containing the scissile bond and the interdomain interactions that clamp helix 3 in the HD must be disrupted for TACE to access the S2 site.

Alternatively, ligand binding could induce conformational exposure of the S2 site allosterically, without exerting a pulling force on the receptor. Because the interface between the LNR and HD domains is extensive (burying ˜3000 Å²) it seems less likely that binding of ligand to the EGF repeat region almost 1000 amino acids away from the S2 site can provide the required energy to disrupt this interface by an allosteric mechanism. However, even though engineered soluble ligands act as antagonists in vertebrates and the fly (Sun et al. 1997, Development 124: 3439-2348; Varnum-Finney et al. 2000, J cell Sci 113, 23, 4313-4318) the worm does produce a natural secreted ligand that activates Notch (Chen et al. 2004, Devel Cell 6: 183-192). This observation suggests that worms have either evolved an adaptor molecule that stably tethers the secreted ligand to allow for the development of force, or that ligands can indeed induce exposure of the metalloprotease site via an allosteric, rather than force-dependent, mechanism.

Our structure of the NRR also provides insight into the ligand-independent activation of NOTCH1 caused by leukemia-associated HD domain mutations (Weng et al. 2004, Science 306, 269-271; Malecki et al. 2006, Mol Cell Biol 26, 4642-4651). The most common mutations lie within the hydrophobic core of the HD (FIG. 6c,d), and cause increased sensitivity to S2 cleavage and decreased NRR stability. These hydrophilic and non-conservative amino acid substitutions within the hydrophobic core of the HD domain likely act by partially or completely unfolding the domain, thereby destroying the small hydrophobic pocket that houses the S2 site and preventing the LNRs from protecting it (FIG. 6d). This possibility is supported by biochemical data showing that mutations of this class destabilize the S1-cleaved HD domain of NOTCH1. A second, less frequent group of mutations, which consist of insertions of 12-14 amino acid residues immediately N-terminal of the S2 site, likely displace the NRR and leave the S2 site unprotected. The autoinhibited conformation reported here now opens a new avenue for the development of therapeutics that target Notch signaling, enabling a structure-based search for small molecules or antibodies designed either to stabilize (e.g. for treatment of T-ALL) or disrupt the restrained conformation of the receptor.

The foregoing written specification is considered to be sufficient to enable one skilled in the art to practice the invention. The present invention is not to be limited in scope by examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description and fall within the scope of the appended claims. The advantages and objects of the invention are not necessarily encompassed by each embodiment of the invention.

What is claimed is:

1. An isolated peptide comprising a human LNR-HD domain having at least one deletion or substitution from a native LNR-HD domain, wherein the isolated peptide has a native conformation.

2-6. (canceled)

7. The isolated peptide of claim 1, wherein the isolated peptide has one or more mutations associated with T-cell acute lymphoblastic leukemia/lymphoma.

8. A method for treating a disease comprising administering to a subject in need of such a treatment an effective amount for treating the disease of a nucleic acid of at least 15 nucleotides which hybridizes under stringent conditions to a LNR-HD domain.

9. The method of claim 8, wherein the LNR-HD domain contains one or more mutations associated with T-cell acute lymphoblastic leukemia/lymphoma.

10. The method of claim 8, wherein the nucleic acid to be administered is an siRNA, antisense RNA or antisense DNA.

11. (canceled)

12. The method of claim 8, where the disease is T-cell acute lymphoblastic leukemia/lymphoma.

13-19. (canceled)

20. A compound comprising an LNR-HD antibody or fragment thereof that binds to an isolated peptide comprising a human LNR-HD domain.

21. The compound of claim 20, wherein the LNR-HD antibody or fragment thereof does not bind to a region of the hNotch receptor other than the LNR-HD domain.

22. (canceled)

23. A method for treating a disease comprising administering to a subject in need of such a treatment an effective amount for reducing Notch activity of an LNR-HD antibody or fragment thereof.

24. The method of claim 23, wherein the LNR-HD antibody or fragment thereof binds to an LNR-HD domain that contains one or more mutations associated with T-cell acute lymphoblastic leukemia/lymphoma.

25. The method of claim 23, wherein the LNR-HD antibody or fragment thereof binds to the native hNotch1 or hNotch2 receptor and abolishes or attenuates the function of the native hNotch1 or hNotch2 receptor.

26-28. (canceled)

29. The method of claim 23, where the disease is T-cell acute lymphoblastic leukemia/lymphoma.

30-39. (canceled)

40. A method for producing an LNR-HD antibody comprising contacting an antibody producing cell with an isolated peptide comprising a human LNR-HD domain that is not in the context of the full length hNotch protein, under conditions effective to produce a LNR-HD specific antibody producing cell, and promoting production of the LNR-HD antibody by the antibody producing cell.

41. (canceled)

42. A method for treating a disease comprising administering to a subject in need of such a treatment an effective amount of an isolated peptide or fragment thereof that binds to an isolated peptide comprising a human LNR-HD domain.

43. The method of claim 43, wherein the isolated peptide binds to the native hNotch1 or hNotch2 receptor.

44. The method of claim 42, wherein the native hNotch1 or hNotch2 receptor contains one or more mutations associated with T-cell acute lymphoblastic leukemia/lymphoma.

45-54. (canceled)

55. An isolated nucleic acid encoding a peptide comprising a human LNR-HD domain having at least one deletion or substitution from a native LNR-HD domain, wherein the nucleic acid is cDNA, genomic DNA or RNA.

56. The isolated nucleic acid of claim 55, wherein the LNR-HD domain contains one or more mutations associated with T-cell acute lymphoblastic leukemia/lymphoma.

57. (canceled)

58. A kit comprising a container housing an LNR-HD domain therapeutic and instructions for administering the components in the kit to a subject at risk of or in need of treatment of any disease.

59-61. (canceled)

62. The method of claim 42, wherein the isolated peptide comprising the human LNR-HD domain has at least one deletion or substitution from a native LNR-HD domain.

63. The method of claim 42, wherein the disease is T-cell acute lymphoblastic leukemia/lymphoma.

64. The compound of claim 20, wherein the isolated peptide comprising the human LNR-HD domain has at least one deletion or substitution from a native LNR-HD domain and, wherein the isolated peptide has a native conformation.

65. The compound of claim 20, wherein the LNR-HD domain contains one or more mutations associated with T-cell acute lymphoblastic leukemia/lymphoma.


SEQUENCE LISTING (44 sequences)

SEQ ID NO: 1 - protein, 319 aa, Homo sapiens
MGSSHHHHHHSSGLVPRGSHMAGSENLYFQGEEACELPECQEDAGNKVCSLQCNNHACGWDGGDCSLNFNDPWKNCTQSLQCWKYFSDGHCDSQCNSAGCLFDGFDCQRAEGQCNPLYDQYCKDHFSDGHCDQGCNSAECEWDGLDCAEHVPERLAAGTLVVVVLMPPEQLRNSSFHFLRELSRVLHTNVVFKRDAHGQQMIFPYYGREEELRKHPIKRAAEGWAAPDALLGQVKASLLPGGSEGGRRRRELDPMDVRGSIVYLEIDNRQCVQASSQCFQSATDVAAFLGALASLGSLNIPYKIEAVQSETVEPPPPAQ

SEQ ID NO: 2 - protein, 288 aa, Homo sapiens
MGSSHHHHHHSSGLVPRGSHMAGSENLYFQGEEACELPECQEDAGNKVCSLQCNNHACGWDGGDCSLNFNDPWKNCTQSLQCWKYFSDGHCDSQCNSAGCLFDGFDCQRAEGQCNPLYDQYCKDHFSDGHCDQGCNSAECEWDGLDCAEHVPERLAAGTLVVVVLMPPEQLRNSSFHFLRELSRVLHTNVVFKRDAHGQQMIFPYYGREEELRKHPIKRELDPMDVRGSIVYLEIDNRQCVQASSQCFQSATDVAAFLGALASLGSLNIPYKIEAVQSETVEPPPPAQ

SEQ ID NO: 3 - protein, 290 aa, Homo sapiens
MGSSHHHHHHSSGLVPRGSHMAGSENLYFQGEEACELPECQEDAGNKVCSLQCNNHACGWDGGDCSLNFNDPWKNCTQSLQCWKYFSDGHCDSQCNSAGCLFDGFDCQRAEGQCNPLYDQYCKDHFSDGHCDQGCNSAECEWDGLDCAEHVPERLAAGTLVVVVLMPPEQLRNSSFHFLRELSRVLHTNVVFKRDAHGQQMIFPYYGREEELRKHPIRRRRELDPMDVRGSIVYLEIDNRQCVQASSQCFQSATDVAAFLGALASLGSLNIPYKIEAVQSETVEPPPPAQ

SEQ ID NO: 4 - protein, 285 aa, Homo sapiens
MGSSHHHHHHSSGLVPRGSHMAGSENLYFQGEEACELPECQEDAGNKVCSLQCNNHACGWDGGDCSLNFNDPWKNCTQSLQCWKYFSDGHCDSQCNSAGCLFDGFDCQRAEGQCNPLYDQYCKDHFSDGHCDQGCNSAECEWDGLDCAEHVPERLAAGTLVVVVLMPPEQLRNSSFHFLRELSRVLHTNVVFKRDAHGQQMIFPYYGREEELRRRRELDPMDVRGSIVYLEIDNRQCVQASSQCFQSATDVAAFLGALASLGSLNIPYKIEAVQSETVEPPPPAQ

SEQ ID NO: 5 - protein, 280 aa, Homo sapiens
MGSSHHHHHHSSGLVPRGSHMAGSENLYFQGEEACELPECQEDAGNKVCSLQCNNHACGWDGGDCSLNFNDPWKNCTQSLQCWKYFSDGHCDSQCNSAGCLFDGFDCQRAEGQCNPLYDQYCKDHFSDGHCDQGCNSAECEWDGLDCAEHVPERLAAGTLVVVVLMPPEQLRNSSFHFLRELSRVLHTNVVFKRDAHGQQMIFPYYGREEELRKHMDVRGSIVYLEIDNRQCVQASSQCFQSATDVAAFLGALASLGSLNIPYKIEAVQSETVEPPPPAQ

SEQ ID NO: 6 - protein, 317 aa, Homo sapiens
MGSSHHHHHHSSGLVPRGSHMAGSENLYFQGEEACELPECQEDAGNKVCSLQCNNHACGWDGGDCSLNFNDPWKNCTQSLQCWKYFSDGHCDSQCNSAGCLFDGFDCQRAEGQCNPLYDQYCKDHFSDGHCDQGCNSAECEWDGLDCAEHVPERLAAGTLVVVVLMPPEQLRNSSFHFLRELSRVLHTNVVFKRDAHGQQMIFPYYGREEELRKHPIKRAAEGWAAPDALLGQVSLLPGGSEGGRRRRELDPMDVRGSIVYLEIDNRQCVQASSQCFQSATDVAAFLGALASLGSLNIPYKIEAVQSETVEPPPPAQ

SEQ ID NO: 7 - protein, 308 aa, Homo sapiens
MGSSHHHHHHSSGLVPRGSHMAGSENLYFQGEEACELPECQEDAGNKVCSLQCNNHACGWDGGDCSLNFNDPWKNCTQSLQCWKYFSDGHCDSQCNSAGCLFDGFDCQRAEGQCNPLYDQYCKDHFSDGHCDQGCNSAECEWDGLDCAEHVPERLAAGTLVVVVLMPPEQLRNSSFHFLRELSRVLHTNVVFKRDAHGQQMIFPYYGREEELRKHPIKRAAEGWASLLPGGSEGGRRRRELDPMDVRGSIVYLEIDNRQCVQASSQCFQSATDVAAFLGALASLGSLNIPYKIEAVQSETVEPPPPAQ

SEQ ID NO: 8 - protein, 308 aa, Homo sapiens
MGSSHHHHHHSSGLVPRGSHMAGSENLYFQGEEACELPECQEDAGNKVCSLQCNNHACGWDGGDCSLNFNDPWKNCTQSLQCWKYFSDGHCDSQCNSAGCLFDGFDCQRAEGQCNPLYDQYCKDHFSDGHCDQGCNSAECEWDGLDCAEHVPERLAAGTLVVVVLMPPEQLRNSSFHFLRELSRVLHTNVVFKRDAHGQQMIFPYYGRAAEGWAAPDALLGQVKASLLPGGSEGGRRRRELDPMDVRGSIVYLEIDNRQCVQASSQCFQSATDVAAFLGALASLGSLNIPYKIEAVQSETVEPPPPAQ

SEQ ID NO: 9 - protein, 150 aa, Homo sapiens
MDQPENLAEGTLVIVVLMPPEQLLQDARSFLRALGTLLHTNLRIKRDSQGELMVYPYYGEKSAAMKKQRMTRRSLPGEQEQEVAGSKVFLEIDNRQCVQDSDHCFKNTDAAAALLASHAIQGTLSYPLVSVVSESLTPERTQLEHHHHHH

SEQ ID NO: 10 - protein, 158 aa, Homo sapiens
MHHHHHHGSENLYFQGDQPENLAEGTLVIVVLMPPEQLLQDARSFLRALGTLLHTNLRIKRDSQGELMVYPYYGEKSAAMKKQRMTRRSLPGEQEQEVAGSKVFLEIDNRQCVQDSDHCFKNTDAAAALLASHAIQGTLSYPLVSVVSESLTPERTQL

SEQ ID NO: 11 - protein, 155 aa, Homo sapiens
MHHHHHHGSENLYFQGENLAEGTLVIVVLMPPEQLLQDARSFLRALGTLLHTNLRIKRDSQGELMVYPYYGEKSAAMKKQRMTRRSLPGEQEQEVAGSKVFLEIDNRQCVQDSDHCFKNTDAAAALLASHAIQGTLSYPLVSVVSESLTPERTQL

SEQ ID NO: 12 - protein, 133 aa, Homo sapiens
MHHHHHHGSENLYFQGENLAEGTLVIVVLMPPEQLLQDARSFLRALGTLLHTNLRIKRDSQGELMVYPYYGEVAGSKVFLEIDNRQCVQDSDHCFKNTDAAAALLASHAIQGTLSYPLVSVVSESLTPERTQL

SEQ ID NO: 13 - protein, 137 aa, Homo sapiens
MHHHHHHGSENLYFQGENLAEGTLVIVVLMPPEQLLQDARSFLRALGTLLHTNLRIKRDSQGELMVYPYYGRRRREVAGSKVFLEIDNRQCVQDSDHCFKNTDAAAALLASHAIQGTLSYPLVSVVSESLTPERTQL

SEQ ID NO: 14 - protein, 138 aa, Homo sapiens
MHHHHHHGSENLYFQGENLAEGTLVIVVLMPPEQLLQDARSFLRALGTLLHTNLRIKRDSQGELMVYPYYGRRRRGEVAGSKVFLEIDNRQCVQDSDHCFKNTDAAAALLASHAIQGTLSYPLVSVVSESLTPERTQL

SEQ ID NO: 15 - protein, 283 aa, Homo sapiens
MGSSHHHHHHSSGLVPRGSHMENLYFQGATCLSQYCADKARDGVCDEACNSHACQWDGGDCSLTMENPWANCSSPLPCWDYINNQCDELCNTVECLFDNFECQGNSKTCKYDKYCADHFKDNHCNQGCNSEECGWDGLDCAADQPENLAEGTLVIVVLMPPEQLLQDARSFLRALGTLLHTNLRIKRDSQGELMVYPYYGEKSAAMKKQRMTRRSLPGEQEQEVAGSKVFLEIDNRQCVQDSDHCFKNTDAAAALLASHAIQGTLSYPLVSVVSESLTPERTQ

SEQ ID NO: 16 - protein, 262 aa, Homo sapiens
MGSSHHHHHHSSGLVPRGSHMENLYFQGATCLSQYCADKARDGVCDEACNSHACQWDGGDCSLTMENPWANCSSPLPCWDYINNQCDELCNTVECLFDNFECQGNSKTCKYDKYCADHFKDNHCNQGCNSEECGWDGLDCAADQPENLAEGTLVIVVLMPPEQLLQDARSFLRALGTLLHTNLRIKRDSQGELMVYPYYGEVAGSKVFLEIDNRQCVQDSDHCFKNTDAAAALLASHAIQGTLSYPLVSVVSESLTPERTQL

SEQ ID NO: 17 - DNA, 31 bp, Artificial sequence
  synthetic oligonucleotide
ggaattccatatggcaggctccgagaacctg

SEQ ID NO: 18 - DNA, 31 bp, Artificial sequence
  synthetic oligonucleotide
cgggggcggccgcgtcattcctaggtggtgg

SEQ ID NO: 19 - DNA, 26 bp, Artificial sequence
  synthetic oligonucleotide
gagctgcgcaagcacatggacgtccg

SEQ ID NO: 20 - DNA, 26 bp, Artificial sequence
  synthetic oligonucleotide
ctcgacgcgttcgtgtacctgcaggc

SEQ ID NO: 21 - DNA, 25 bp, Artificial sequence
  synthetic oligonucleotide
ctgggccaggtgtcgctgctccctg

SEQ ID NO: 22 - DNA, 25 bp, Artificial sequence
  synthetic oligonucleotide
gacccggtccacagcgacgagggac

SEQ ID NO: 23 - DNA, 20 bp, Artificial sequence
  synthetic oligonucleotide
ctacggccgcgccgccgagg

SEQ ID NO: 24 - DNA, 20 bp, Artificial sequence
  synthetic oligonucleotide
gatgccggcgcggcggctcc

SEQ ID NO: 25 - DNA, 21 bp, Artificial sequence
  synthetic oligonucleotide
gggctgggcctcgctgctccc

SEQ ID NO: 26 - DNA, 21 bp, Artificial sequence
  synthetic oligonucleotide
cccgacccggagcgacgaggg

SEQ ID NO: 27 - DNA, 24 bp, Artificial sequence
  synthetic oligonucleotide
gaggagctgcgccggcggagggag

SEQ ID NO: 28 - DNA, 24 bp, Artificial sequence
  synthetic oligonucleotide
ctcctcgacgcggccgcctccctc

SEQ ID NO: 29 - DNA, 50 bp, Artificial sequence
  synthetic oligonucleotide
aattccatatggagaacctgtattttcagggcgccacctgtctgagccag

SEQ ID NO: 30 - DNA, 35 bp, Artificial sequence
  synthetic oligonucleotide
actgaggtcttgcgtgagtcattcctaggtggtgg

SEQ ID NO: 31 - DNA, 35 bp, Artificial sequence
  synthetic oligonucleotide
gtacccctattatggtgaggtggctggctctaaag

SEQ ID NO: 32 - DNA, 35 bp, Artificial sequence
  synthetic oligonucleotide
catggggataataccactccaccgaccgagatttc

SEQ ID NO: 33 - DNA, 43 bp, Artificial sequence
  synthetic oligonucleotide
gtacccctattatggtcgccgtcgccgtgaggtggctggctct

SEQ ID NO: 34 - DNA, 42 bp, Artificial sequence
  synthetic oligonucleotide
catggggataatccagcggcagcggcactccaccgaccgaga

SEQ ID NO: 35 - DNA, 45 bp, Artificial sequence
  synthetic oligonucleotide
gtacccctattatggtcgccgtcgccgtggcgaggtggctggctc

SEQ ID NO: 36 - DNA, 45 bp, Artificial sequence
  synthetic oligonucleotide
catggggataataccagcggcagcggcaccgctccaccgaccgag

SEQ ID NO: 37 - DNA, 33 bp, Artificial sequence
  synthetic oligonucleotide
ggaattccatatggaccaacctgagaacctggc

SEQ ID NO: 38 - DNA, 30 bp, Artificial sequence
  synthetic oligonucleotide
actgaggtcttgcgtgagtcggagctcgcc

SEQ ID NO: 39 - DNA, 49 bp, Artificial sequence
  synthetic oligonucleotide
acggatccgagaacctgtactttcaaggtgaccaacctgagaacctggc

SEQ ID NO: 40 - DNA, 32 bp, Artificial sequence
  synthetic oligonucleotide
gaggtcttgcgtgagtcgagattgagctcgcc

SEQ ID NO: 41 - DNA, 47 bp, Artificial sequence
  synthetic oligonucleotide
ggaattccatatgcaccaccaccaccaccacggatccgagaacctgt

SEQ ID NO: 42 - DNA, 17 bp, Artificial sequence
  synthetic oligonucleotide
tcgagattgagctcgcc

SEQ ID NO: 43 - DNA, 32 bp, Artificial sequence
  synthetic oligonucleotide
cctgtactttcaaggtgagaacctggcagaag

SEQ ID NO: 44 - DNA, 32 bp, Artificial sequence
  synthetic oligonucleotide
ggacatgaaagttccactcttggaccgtcttc